SEQUENCE LISTING (7 sequences)

SEQ ID NO: 1 - DNA, 52 bp, Artificial Sequence
  Synthetic polynucleotide
gcatacgtctgtacaagggatctggaggatctggaatcagtctgattgcggc

SEQ ID NO: 2 - DNA, 31 bp, Artificial Sequence
  Synthetic polynucleotide
gcatacgaattcttaccgccgctccagaatc

SEQ ID NO: 3 - DNA, 35 bp, Artificial Sequence
  Synthetic polynucleotide
gcatacgtcggatcccatggtgagcaagggcgagg

SEQ ID NO: 4 - DNA, 27 bp, Artificial Sequence
  Synthetic polynucleotide
gcatacgtctctacagctcgtccatgc

SEQ ID NO: 5 - DNA, 1311 bp, Artificial Sequence
  Synthetic polynucleotide
atgcggggttctcatcatcatcatcatcatggtatggctagcatgactggtggacagcaaatgggtcgggatctgtacgacgatgacgataaggatcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatctggaggatctggaatcagtctgattgcggcgttagcggtagatcgcgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgggtacggacgatcgcgtaacgtgggtgaagtcggtggatgaagccatcgcggcgtgtggtgacgtaccagaaatcatggtgattggcggcggtcgcgtttatgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcggtaa

SEQ ID NO: 6 - protein, 436 aa, Artificial Sequence
  Synthetic polypeptide
MRGSHHHHHHGMASMTGGQQMGRDLYDDDDKDPMVSKGEELFTGVVPILVELDGDVNGHKFSVSGEGEGDATYGKLTLKFICTTGKLPVPWPTLVTTFSYGVMVFARYPDHMKQHDFFKSAMPEGYVQERTIFFKDDGNYKTRAEVKFEGDTLVNRIELKGIDFKEDGNILGHKLEYNFNSHNVYIMADKQKNGIKANFKIRHNIEDGGVQLADHYQQNTPIGDGPVLLPDNHYLSIQSKLSKDPNEKRDHMVLLEFVTAAGITLGMDELYKGSGGSGISLIAALAVDRVIGMENAMPWNLPADLAWFKRNTLNKPVIMGRHTWESIGRPLPGRKNIILSSQPGTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 7 - protein, 8 aa, Artificial Sequence
  Synthetic polypeptide
DPKKKRKV

(19) United States
(12) Patent Application Publication
(10) Pub. No.: US 2012/0115128 A1
(43) Pub. Date: May 10, 2012

(54) SELECTIVE PROTEIN LABELING

(75) Inventor: Larry Miller, Chicago, IL (US)

(73) Assignee: THE BOARD OF TRUSTEES OF THE UNIVERSITY OF ILLINOIS, Urbana (IL)

(21) Appl. No.: 13/318,517

(22) PCT Filed: May 7, 2010
(86) PCT No.: PCT/US2010/034133

§ 371 Date: Jan. 23, 2012

Related U.S. Application Data
(60) Provisional application No. 61/176,393, filed on May 7, 2009.

(51) Int. Cl. C12Q 1/02; C12Q 1/68; C12Q 1/18

(52) U.S. Cl. 435/6.1; 435/32; 435/29

(57) ABSTRACT

The present invention is related to methods of detecting protein-protein interactions in living cells, as well as detecting the formation and/or inhibition of protein-protein interactions in cells.

### CROSS-REFERENCE TO RELATED APPLICATIONS

This application claims the priority benefit under 35 U.S.C. §119(e) of U.S. Provisional Application No. 61/176,393, filed on May 7, 2009, the disclosure of which is incorporated herein by reference in its entirety.

### STATEMENT OF GOVERNMENT INTEREST

This invention was made with government support under Grant Number NIGMS R01GM081030, awarded by The National Institutes of Health (NIH). The government has certain rights in the invention.

### FIELD OF THE INVENTION

The present invention is related to methods of detecting protein-protein interactions in living cells, as well as detecting the formation and/or inhibition of protein-protein interactions in cells.

### BACKGROUND

Current understanding of cellular biology and the molecular interactions that lead to the development and progression of diseases is primarily based upon easily characterized static models. However, biomolecules, particularly proteins, interact transiently within multi-component complexes to control biological processes such as the cell cycle, motility or immune response (Gavin et al. Nature 440:631-636 (2006)). In order to understand the dynamic organization of protein complexes, new experimental tools will be required that incorporate 1) specific, simultaneous labeling of multiple, selected proteins in living cells or tissues with high signal-to-background ratio contrast agents; 2) imaging instrumentation that allows real-time, multiplexed detection of protein location, interaction state, and interaction stoichiometry; and 3) live cell and/or tissue model systems that reasonably approximate in vivo conditions, and are amenable to physiologically relevant perturbations that are hypothesized to alter transient protein organization (Schultz et al. Chembiochem 6:1323-1330 (2005)).

Microscopic imaging of protein-protein interactions in living cells currently relies extensively on fluorescence resonance energy transfer (FRET) between cyan fluorescent protein (CFP) and yellow fluorescent protein (YFP) (J. Zhang, R. E. Campbell, A. Y Ting, R. Y Tsien Nat. Rev. Mol. Cell. Biol. 2002 3, 906-918). In FRET, a donor fluorophore (e.g., CFP) is excited by incident light, and the excited state energy from the donor is non-radiatively transferred to a nearby (≧5 nm) acceptor fluorophore (e.g., YFP). Fluorescence microscopy dynamically detects the spatial distribution of interactions between CFP and YFP fusion proteins in living cells as a decrease in CFP emission and concomitant increase in YFP emission. However, FRET-based imaging of protein-protein interactions using CFP and YFP is problematic for several reasons: 1) fluorescent protein spectra are broad and overlapping, necessitating multiple control measurements and corrective algorithms to normalize for the dependence of FRET on the concentration of donors and acceptors; 2) CFP and YFP exhibit a FRET dynamic range of less than 5-fold, severely limiting the signal-to-background ratio of FRET imaging measurements; and 3) only a single FRET interaction (CFP/YFP, or more rarely, green fluorescent protein/monomeric red fluorescent protein) can be easily resolved microscopically, eliminating the ability to simultaneously observe more than one interaction in a single cell (J. Zhang, R. E. Campbell, A. Y Ting, R. Y Tsien Nat. Rev. Mol. Cell. Biol. 2002 3, 906-918; A. W. Nguyen, P. S. Daugherty Nat. Biotechnol. 2005, 23, 355-360). Collectively, these limitations make quantitative, real-time observation of protein-protein interactions extremely difficult, if not impossible.

The use of lanthanide complexes (LC) with long (i.e., millisecond) luminescent lifetimes as FRET donors coupled with time-resolved detection methods is well established for detecting protein-protein interactions in vitro at high signal-to-background ratio (P. Selvin Ann Rev. Biophys. 2002, 31, 275-302). However, to date, lanthanide complexes have not been used as FRET donors in live cell imaging or spectroscopy because methods are needed to deliver lanthanide probes from culture medium to the interior of cells, and methods are needed to specifically append the probe to a protein or subcellular structure of interest.

Thus, there exists a need in the art to visualize multiple protein-protein interactions in living cells, as well as detect the formation and/or inhibition of protein-protein interactions in cells in a high-throughput manner.

### SUMMARY OF THE INVENTION

Herein it is disclosed that lanthanide probes, with the dramatic lowering of background achieved through time-gating, provide new microscopic imaging when successfully coupled with cell penetration and molecular targeting and recognition.

A composition is provided comprising a lanthanide complex (LC) and a cell-penetrating peptide (CPP), the LC comprising a chelating moiety in association with a lanthanide and a ligand. As used herein, an LC is a lanthanide ion in a chelating moiety which is in association with a ligand, each as defined herein. The ligand is a polypeptide which binds to a detector polypeptide fused to a target molecule. In one embodiment, the lanthanide is terbium. In another embodiment, the lanthanide is europium. In some aspects, the ligand is trimethoprim (TMP) or a TMP analog. In various aspects, the cell-penetrating peptide is selected from the group consisting of oligo-arginine, a TAT-peptide, and rabies virus glycoprotein.

A method of labeling a first target molecule is also provided, the method comprising the step of contacting the target molecule with a non-toxic concentration of a lanthanide complex (LC) under conditions that allow association of the LC and the first target molecule, wherein the association labels the first target molecule, said LC comprising a ligand and a chelating moiety comprising a lanthanide. In one aspect, the LC associates with a first detector molecule fused to the target molecule and in another aspect, the first detector molecule associates with the LC through a ligand associated with the LC.

The method further contemplates use of a second target molecule, the second target molecule fused to a second detector molecule, the method performed under conditions wherein the second target molecule interacts with the first target molecule. In one aspect, interaction of the first target molecule with the second target molecule brings the LC and the second detector molecule in to sufficient proximity to permit an energy exchange from the LC to the second detector molecule and wherein energy transfer to the second detector molecule permits detection of the first target molecule.

Methods are contemplated wherein labeling is in a host cell, or in the alternative, labeling is on the surface of a host cell.

In another aspect, the method provided further comprises the step of culturing the host cell transformed or transfected with a first nucleic acid construct comprising a promoter element operatively-linked to a nucleic acid encoding a fusion protein, the fusion protein comprising the first target molecule and a first detector molecule under conditions such that a first target molecule/detector molecule fusion is expressed. In another aspect, the method further comprises the step of culturing the host cell transformed or transfected with a second nucleic acid construct comprising a promoter element operatively-linked to a nucleic acid encoding a fusion protein, the fusion protein comprising the second target molecule and a second detector molecule under conditions such that a first target molecule/detector molecule fusion is expressed.

In every variation of the method, it is contemplated, in one aspect, that the LC bound to the fusion protein is detected with a device, the lanthanide is terbium and/or the lanthanide is europium.

In one aspect, of the method presence of the first target molecule is detected by fluorescence microscopy.

The method provided includes an embodiment wherein the second detector molecule is a fluorophore, and in certain aspect, the fluorophore is a fluorescent protein.

The methods in each embodiment contemplate various aspects wherein the first detector molecule is a dihydrofolate reductase and the ligand in the LC is trimethoprim (TMP) or the first detector molecule is a dihydrofolate reductase and the ligand in the LC is a trimethoprim (TMP) analog.

In yet another embodiment of each variation of the method, the first target molecule is selected from the group consisting of a protein, a protein domain, and a peptide and the second target molecule is selected from the group consisting of a protein, a protein domain, and a peptide.

In a further embodiment of each variation of the method wherein labeling is in a host cell, the LC further comprises a cell-penetrating peptide (CPP). In some aspects, the CPP is selected from the group consisting of oligo-arginine, a TAT-peptide, and rabies virus glycoprotein.

### BRIEF DESCRIPTION OF THE FIGURES

FIG. 1 shows: A) Representation of selective protein labeling. B) Schematic of intermolecular LRET between Tb³⁺-complex-tagged donor and GFP-tagged acceptor. C) Overlay of Tb³⁺ emission spectrum and GFP and Rhodamine excitation and emission spectra. With multiple emission maxima, Tb³⁺ can serve as an LRET donor to different acceptors, allowing simultaneous detection of multiple protein-protein interactions. Eu³⁺ also has multiple emission peaks, offering additional options for multiplexed analysis. D) Time-resolved microscopy detects LRET signals at high signal-to-background ratio by rejecting sample autofluorescence and directly excited acceptor fluorescence. Analysis of either donor or sensitized acceptor luminescent lifetime allows quantification of protein-protein interaction stoichiometry.

FIG. 2 shows protein-targeted LCs. The proposed LCs have a general structure consisting of a protein targeting ligand (trimethoprim, TMP) linked to a sensitized chelating complex (upper left). A series of sensitized LCs linked to TMP have been synthesized based on polyamino-carboxylate (upper right) or 1,4,7,10-tetra-azacyclododecane-N,N′,N″,N′″-tetraacetic acid (upper left). When complexed with Tb³⁺ or Eu³⁺ and illuminated with UV light (ca. 340 nm), the sensitizer (carbostyril 124) is excited and energy is transferred intramolecularly to emittive levels of the chelated lanthanide ion, resulting in characteristic lanthanide luminescence. The TMP moiety confers affinity for E. coli DHFR. A variety of sensitized LCs have been described in the literature. In some cases, the chelating moiety is separate from the sensitizer, and in other cases the sensitizing and chelating moieties are integral. (Rows 3-5) TMP shown linked to various LCs that have been reported, including terperydine (Row 2).

FIG. 3 depicts a portion of an LC of the type where the sensitizing moiety and the chelating moiety are combined. (Top) terpyridine (Hovinen et al., Bioconjugate Chem. 2009, 20:404-422); (Middle) 2-hydroxyisophthalamide (Petoud et al., J Am Chem Soc 2003, 125:13324-13325; see also WO 2008/063721, incorporated herein by reference in its entirety); (Bottom) Helicate. Here, three chelating moieties complex two europium ions to form the luminescent complex (Vandevyver et al., Chemical Communications 2007:1716-1718).

FIG. 4 depicts intracellular delivery and specific protein targeting of an LC linked to a cell penetrating peptide (CPP). A heterodimeric conjugate of trimethoprim (TMP) linked to LC is covalently linked to the N-terminus of a CPP via a disulfide bond. A Quencher molecule is shown linked to the C-terminus of the peptide. Upon addition to cell culture medium, the LC-CPP conjugate is transduced into the interior of the cells via an endocytic mechanism. LC-CPP molecules that escape endosomes into the cytosol are cleaved via reduction of the disulfide bond. Free LCs are separated from the quencher moiety and rendered luminescent, where they bind to recombinant DHFR fusion proteins. LC-CPPs that remain trapped in endosomes are quenched and thus non-luminescent.

FIG. 5 depicts normalized emission spectra of terbium-complexed TMP-LCs. a) TMP-Lumi4 (λₑₓ=339 nm); b) TMP-cTTHA (λₑₓ=343 nm); c) TMP-cDTPA (λₑₓ=343 nm).

FIG. 6 shows intramolecular, time-resolved, fluorescence resonance energy transfer (TR-FRET) between eDHFR-bound TMP-LCs and GFP. Increasing concentrations of purified eDHFR-GFP were titrated against a constant concentration (20 nM) of each compound. Sensitized GFP emission (520 nm) was detected after a time delay of 100 μs, upon pulsed excitation with near-UV light (ca. 340 nm): diamonds, TMP-Lumi4; circles, TMP-cTTHA; x's, TMP-cDTPA. Addition of 1 μM TMP reduced the signal, confirming FRET (shown for TMP-cTTHA, squares). Lines represent non-linear least squares fit to the data.

FIG. 7 depicts time-resolved microscopy of NIH3T3 cells treated with TMP-LCs. a) Overlay of bright field and prompt fluorescence (λₑₓ=490 nm) images of cells transiently expressing nucleus-localized CFP and plasma membrane-localized eDHFR. b) Inverse, time-resolved fluorescence image of cells in a) showing non-specific luminescence. Cells were incubated 20 hours in media containing TMP-cTTHA (100 μm), washed with PBS, mounted in media without compound, and imaged in time-resolved mode (λₑₓ=ca. 350 nm, λₑₘ=550 nm, delay=80 μs, exposure time=1420 μs, exposure cycles=660). c) Overlay image of cells transiently expressing nucleus-localized CFP and cell surface-localized eDHFR. d) Inverse, time-resolved fluorescence image of cells in c) showing membrane luminescence in transfected cell. Cells were incubated in media containing 1 μM TMP-Lumi4 (10 min.), washed, and imaged as in b).

FIG. 8 depicts the principle of time-resolved detection. Pulsed light (width=T) excites samples. Longlifetime probe luminescence is detected during an interval (T₀) after a short delay (Δt) allows decay of scattering and autofluorescence background. Multiple excitation/emission cycles (period=T′) are integrated to increase signal.

FIG. 9 depicts cytosolic delivery and specific, intracellular labeling of eDHFR fusion proteins with TMP-Lumi4 visualized by time-resolved fluorescence microscopy. (a-c) Micrographs: BF, bright field; CW, continuous wave fluorescence (λₑₓ=480±20 nm, λₑₘ=535±25 nm); TRF, time-resolved fluorescence (λₑₓ=365 nm, λₑₘ>400 nm, Δt=100 μs); TRF+TMP, 20 min. after addition of TMP (final conc.=10 μM) to growth medium; TR-FRET, time-resolved FRET (λₑₓ=365 nm, λₑₘ=520±10 nm, Δt=100 μs). TRF and TRF,+TMP (negative control) images were adjusted to identical contrast levels. Scale bars, 10 μm. (a) NIH3T3 cells expressing nucleuslocalized CFP and plasma membrane-localized eDHFR. Cells were loaded with TMP-Lumi4 (15 μM) by SLO permeabilization (10 min.). (b) MDCK cells expressing nucleus-localized CFP and nucleus-localized eDHFR. Cells were loaded with TMP-Lumi4 (15 μM) by SLO permeabilization (10 min.). (c) NIH3T3 cells expressing GFP-eDHFR, loaded by pinocytosis (10 min.) of culture medium containing TMP-Lumi4 (50 μM) followed by osmotic lysis of pinosomes.

FIG. 10 depicts time-resolved imaging of terbium-sensitized GFP emission reveals interaction between TMP-Lumi4-labeled ZO-1/PDZ1-eDHFR and GFP-cldn1/tail in living MDCK cells. (a-c) Micrographs: BF, bright field; CW, continuous wave fluorescence (λₑₓ=480±20 nm, λₑₘ=535±25 nm); TRF, time-resolved fluorescence (λₑₓ=365 nm, λₑₘ>400 nm, Δt=100 μs); TRFRET, time-resolved FRET (λₑₓ=365 nm, λₑₘ=520±10 nm, Δt=100 μs); TR-FRET+TMP, after addition of TMP (final concentration=10 μM) to growth medium at indicated time-points. TRFRET and TR-FRET,+TMP (negative control) images were adjusted to identical contrast levels. Scale bars, 10 μm. (a) MDCK cells co-expressing ZO-1/PDZ1-eDHFR and GFP-cldn1/tail. Cells were loaded with TMP-Lumi4 (15 μM) by SLO permeabilization (10 min.). (b) MDCK cells coexpressing ZO-1/PDZ1-eDHFR and GFP-cldn1/tail. Cells were loaded by pinocytosis (10 min.) of culture medium containing TMP-Lumi4 (50 μM) followed by osmotic lysis of pinosomes. (c) MDCK cells co-expressing ZO-1/PDZ1-eDHFR and GFP-cldn1/tailΔYV. Cells were loaded by pinocytosis (10 min.) of culture medium containing TMP-Lumi4 (50 μM) followed by osmotic lysis of pinosomes.

### DETAILED DESCRIPTION OF THE INVENTION

The present disclosure provides materials and methods for detecting protein-protein interactions. Compositions provided by the present disclosure comprise a LC, and optionally further comprise a cell penetrating peptide. As used herein, a lanthanide complex (LC) is a lanthanide ion in a chelating moiety in association with a ligand, each as defined below.

### Lanthanides

Lanthanide luminescence offers several advantages for fluorescence-based biological assays: 1) large Stoke's shifts (greater than 150 nm) and multiple, narrow emission bands (less than 10 nm at half-maximum) allow efficient spectral separation of emission signals; 2) long luminescence lifetimes (micro- to millisecond) enable time-resolved detection methods to remove scattering and autofluorescence background; and 3) relative insensitivity to photobleaching allows for prolonged detection (Pandya et al. Dalton Trans 2757 (2006)). Terbium and europium probes typically incorporate a metal ion into an organic chelating ligand that contains a sensitizing chromophore. When excited with near-UV light in the absorption band, the chromophore transfers energy via intersystem crossing to the triplet excited state and intramolecular transfer to the emissive level of the chelated metal (Pandya et al. Dalton Trans 2757 (2006); Hemmila et al. J Fluoresc 15: 529 (2005)). Direct conjugation of lanthanide probes to antibodies, oligonucleotides and proteins has enabled the development of sensitive, time-resolved fluorescence resonance energy transfer (TR-FRET) assays of biomolecular interactions in purified biochemical preparations, cellular extracts, and on cell surfaces (Cha et al. Nature 402: 809 (1999); Ghose et al. Journal of Alloys and Compounds 451: 35 (2008); Jia et al. Analytical Biochemistry 356: 273 (2006); Maurel et al. Nature Methods 5: 561 (2008); Maurel et al. Analytical Biochemistry 329: 253 (2004); Trinquet et al. Molecular Biosystems 2: 381 (2006)).

For time-resolved fluorometry assay systems, luminescent LCs must fulfill several requirements: 1) the molecule has to be chemically stable; 2) the molecule must exhibit good brightness (the product of extinction coefficient and quantum yield of emission); 3) the excitation wavelength has to be as high as possible, preferably over 330 nm; 4) the luminescence decay time has to be long (>10 microseconds); 5) the complex should be readily soluble in water; 6) it must be possible to link the complex to biomolecules without disrupting any of the aforementioned properties or the biological activity of the labeled biomolecule.(Hovinen et al., Bioconjugate Chem. 2009, 20:404-422). Decades-long efforts by many researchers have yielded many LCs that meet some of the above criteria. A considerably smaller subset of existing LCs meet all of the listed criteria.

Structurally, LCs can be divided into two categories: 1) those consisting of an organic chelating complex covalently linked to a distinct organic chromophore that serves as a sensitizer; and 2) those that consist of aromatic substituents that serve to both complex the lanthanide ion and sensitize luminescence.

Scheme 1 (below) shows a schematic of protein-targeted LCs of the first type, in this case based on the 1,4,7,10-tetraazacyclododecane (cyclen) scaffold. In this case, the putative molecule contains five variable elements: 1) the cyclen moiety covalently linked to two or more pendant arms, which serves to complex 2) the lanthanide ion; 3) the sensitizer or antenna that serves to absorb excitation light; 4) a linker of variable length and atomic composition that serves to separate the chelator/sensitizer from 5) the ligand (TMP, in this case). In one aspect, the linker is attached to an amine of the cyclen moiety or via linkage to a methylene group on cyclen. The lanthanide ion, in various aspects, is any one of the lanthanide series. Accordingly, in aspects of the present disclosure, the lanthanide ion is lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

The pendant groups and sensitizers can be varied, and several non-limiting embodiments are shown in the figure. The schematic depicts: A) ligand-coupled, sensitized LC based on the cyclen chelating scaffold; B-F) Sensitizers: B) carbostyril 124 analogs(Li et al., J Am Chem Soc 1995, 117:8132-8138; Li et al., Bioconjugate Chemistry 1997, 8:127-132); C) aminoquinoline (Hess et al., J Phys Chem A 2008, 112:2397-2407); D) Coumarin (Hess et al., J Phys Chem A 2008, 112:2397-2407); E) aza(thia)xanthone (X═O, azaxanthone; X═S, azathiaxanthone) (Kielar et al., Org Biomol Chem 2008, 6:2256-2258); F) tetraazatriphenylene (Poole et al., Org Biomol Chem 2005, 3:1013-1024).

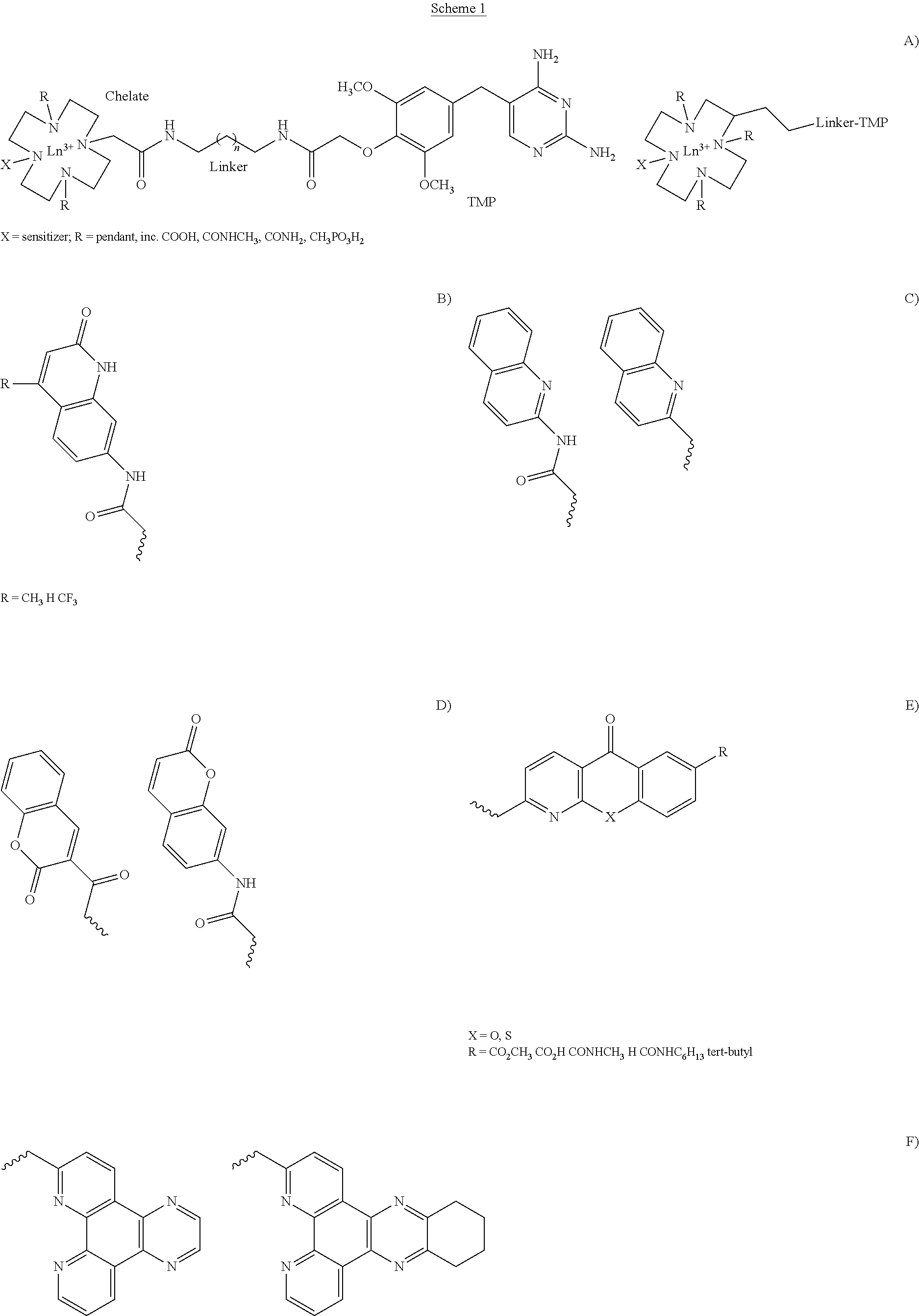

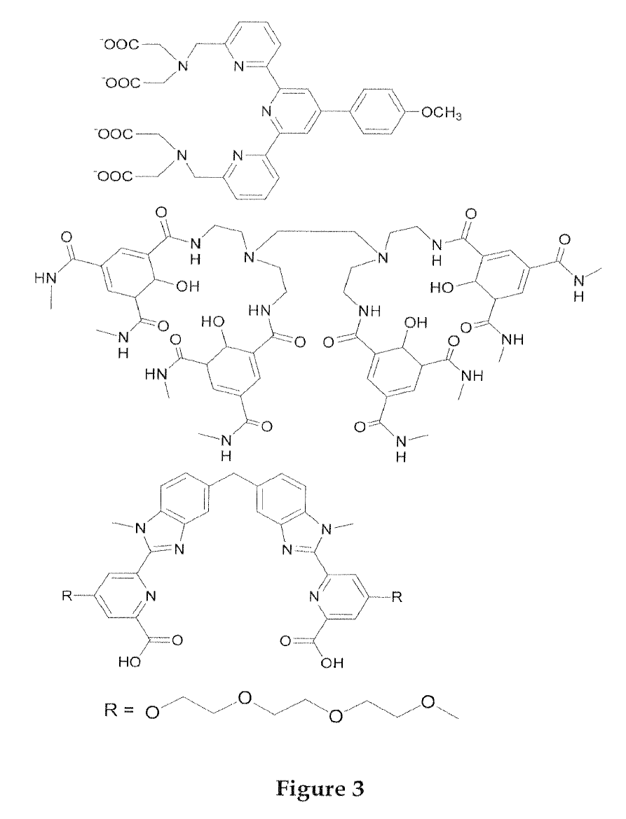

LCs of the second type (where the sensitizing moiety and chelating moiety are combined) are shown in FIG. 3.

LCs have been extensively developed as luminescent probes for in vitro bioassays and, to a much more limited extent, as cellular imaging agents (Pandya et. al., Dalton Trans. 2757 (2006), the subject matter of which is incorporated herein in its entirety). Versatile cellular imaging probes must diffuse readily into cells from culture medium, partition only to the desired sub-cellular compartment, organelle or protein target of interest, and be easily detected using fluorescence microscopy. Individual proteins can be selectively labeled with cell-permeable luminescent probes using one of a number of ligand-receptor protein labeling schemes (Miller et al. Curr. Opinion Chem. Biol. 9: 56 (2005)). One such labeling scheme leverages the strong (K_{D}=approximately 1 nM), orthogonal non-covalent interaction between TMP and Esherichia coli dihydrofolate reductase (eDHFR) (Miller et. al., Nat. Methods 2: 255 (2005); Calloway et. al., Chembiochem 8: 767 (2007)).

### Reporters of Protein Interaction—Studying Protein-Protein Interactions with LRET Microscopy

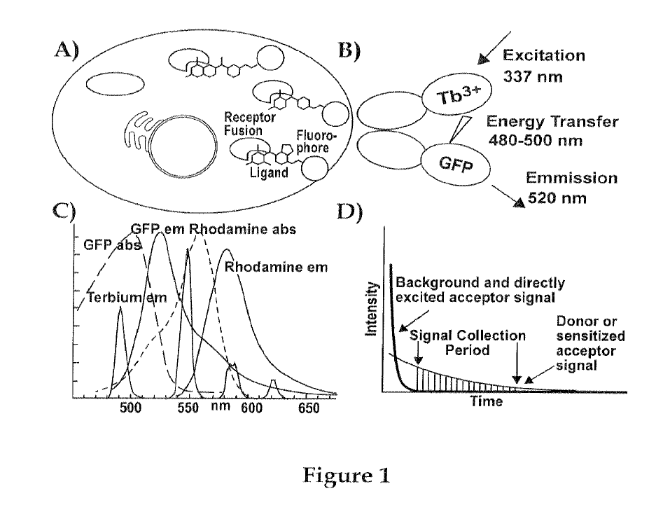

Fluorescence resonance energy transfer (FRET) microscopy is used to image transient protein-protein interactions in living cells. In FRET, a donor fluorophore is excited by incident light, and the excited state energy from the donor is transferred to a nearby (<10 nm) acceptor fluorophore. By using optical microscopy to detect FRET between fusions of green fluorescent protein variants (GFPs) to interacting proteins expressed in cells, it is possible to obtain spatial and temporal information about the binding and interaction of the protein fusions (Giepmans et al. Science 312, 217-224 (2006)). However, the use of GFPs for FRET requires multiple measurements to correct for spectral overlap between donor and acceptor emission and to normalize for the dependence of FRET on the concentration of donors and acceptors (Jares-Erijman et al. Nat Biotechnol 21, 1387-1395 (2003)). Furthermore, currently only a single GFP FRET interaction can be resolved (CFP/YFP, or GFP/RFP), eliminating the ability to simultaneously analyze more than one interaction in a single cell. Long luminescent lifetimes (ca. msec) make LCs optimal donors for FRET (as the emission of lanthanides is not technically fluorescence, the technique is called luminescence resonance energy transfer, or LRET). Because the fluorescence of an organic small molecule or fluorescent protein acceptor has a lifetime of nanoseconds, any long-lifetime acceptor fluorescence is due solely to energy transfer from the LC donor (Selvin et al., Annu Rev Biophys Biomol Struct 31: 275-302 (2002)). Time-resolved, LRET microscopy can be used to image and quantify the transient interactions between a protein coupled to a lanthanide donor (for example and without limitation, by the TMP-eDHFR method) and one or more proteins labeled with GFP or an organic fluorophore (FIG. 1B-D). Analysis of donor and acceptor emission decay data yields the relative concentrations of interacting and non-interacting protein species. The combination of selective protein labeling with LC probes and LRET will make it possible to dynamically detect and stoichiometrically quantify multiple protein-protein interactions in live cells with unprecedented sensitivity and spatio-temporal resolution.

In one set of embodiments, the invention is used to detect the interaction of target proteins. In one aspect, a first target protein is fused to a first detector protein, and a second target protein is fused to a second detector protein using nucleic acid constructs and methods as set forth herein, such that the first target protein and the second target protein are both expressed in a host cell. In certain aspects, the first target protein associates with the second target protein, thereby bringing the first and second target proteins in close proximity. In these aspects, the first detector protein associates with a LC through the ligand component of the LC. The association of the LC with the first detector protein causes an interaction between the LC and the second detector protein resulting in FRET.

In some aspects, the first detector protein is a DHFR. In further aspects, the second detector protein is a fluorescent protein. In still further aspects, the LC comprises TMP.

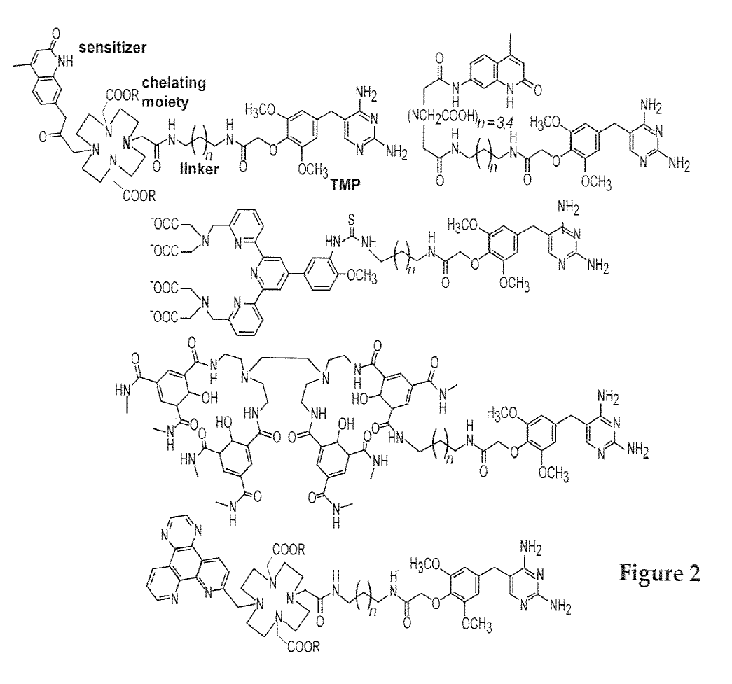

In another aspect, methods of imparting lanthanide luminescence to proteins for in vitro TR-FRET-based assays and live cell imaging applications are provided. In one apect, the method entails the design and synthesis of an LC that associates with, in some aspects, E. coli dihydrofolate reductase (eDHFR) fusion proteins via entry into cells and subsequent, high-affinity (K_{D}=ca. 1 nM), ligand-eDHFR interaction. In some aspects, the ligand is trimethoprim (TMP) The eDHFR fusion protein, in various aspects, further comprises a fluorophore or other functional moiety. When added to cells growing in culture, the LC binds specifically to the eDHFR fusion. Heterodimeric molecules consisting of a detector protein binding ligand, for example and without limitation, TMP, linked to a series of luminescent terbium complexes, including carbostyril 124-linked polyaminocarboxylates (cs124-polyaminocarboxylates) and a 2-hydroxyisophthalamide-based complex are prepared (Scheme 2). The specific labeling of eDHFR expressed on the surface of living mammalian cells with LCs is visualized using a sensitive time-resolved, fluorescence microscope capable of rapid image acquisition. A non-limiting selection of TMP-linked LCs is shown in FIG. 2.

In various aspects, other ligands that have been described for selective chemical protein labeling are incorporated into the methods of the present disclosure, such as and without limitation alkyl chloride (which targets dehalogenase, Halo-Tag™, Promega Inc., Madison, Wis.), benzyl guanine and benzyl cytosine derivatives (which targets hAGT) (Covalys, Inc. and New England Biolabs; Keppler et al. Proc Natl Acad Sci USA 101, 9955-9959 (2004)), or synthetic ligation factor (SLF) that binds non-covalently to a mutant (F36V) of FKBP12 (Invitrogen, Inc.). This alternative strategy has the added benefit that it yields an additional selective LC protein label, thereby enabling simultaneous labeling of two different proteins in a single cell, i.e., one protein is labeled with a first LC and a second protein is labeled with a second LC. In one aspect, for example and without limitation, a first protein is in a terbium-complexed LC and a second protein is in a europium-complexed LC.

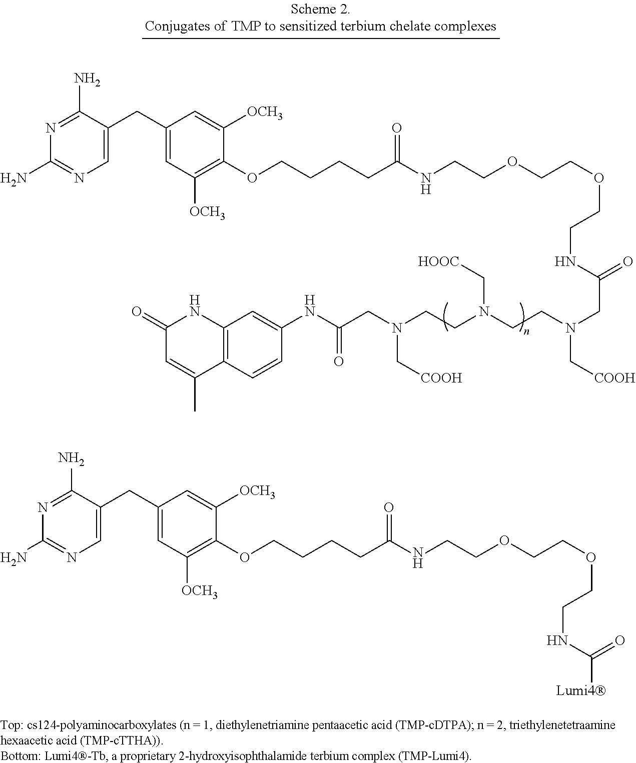

The specific, high affinity (K_{D}=ca. 1 nM) interaction between TMP and eDHFR has been exploited to develop the LigandLink™ Universal Labeling technology (Active Motif, Inc., Carlsbad, Calif.) that makes it possible to tag eDHFR fusion proteins in wild-type mammalian cells with cell-permeable TMP-fluorophore conjugates (Calloway et al., Chembiochem 2007, 8, 767; Miller et al., Nature Methods 2005, 2, 255). The TMP-eDHFR interaction has been utilized for labeling proteins with lanthanide probes. For the first generation of TMP-TCs, terbium complexes that were known to have good brightness (i.e., high extinction coefficients and quantum yields), could be conjugated without disrupting their terbium binding characteristics or luminescence, and could be synthesized relatively easily were selected. Selvin and co-workers have developed and extensively characterized complexes of the chromophore carbostyril-124 (cs124) linked to diethylenetriamine pentaacetic acid (DTPA) and triethylenetetraamine hexaacetic acid (TTHA) (Chen et al., Bioconjugate Chemistry 1999, 10, 311; Li et al., Am Chem Soc 1995, 117, 8132; Li et al., Bioconjugate Chemistry 1997, 8, 127; Xiao et al., Journal of the American Chemical Society 2001, 123, 7067). Terbium complexes of cs124-DTPA and cs124-TTHA have relatively high extinction coefficients (E=ca. 10,000 M⁻¹ cm⁻¹ at 343 nm) and quantum yields in water of 0.32 and 0.40, respectively (Xiao et al., Journal of the American Chemical Society 2001, 123, 7067). Moreover, the complexes exhibited similar brightness and lifetimes when conjugated to peptides or proteins (Chen et al., Bioconjugate Chemistry 1999, 10, 311; Li et al., Bioconjugate Chemistry 1997, 8, 127). Therefore, heterodimers of cs124-DTPA and cs124-TTHA linked to TMP via a 15-atom linker are prepared (Scheme 2), reasoning that the conjugation strategy might preserve the essential characteristics of the parent complexes. Raymond and co-workers reported an extremely bright (E=ca. 28,000 M⁻¹ cm⁻¹ at 354 nm, QY=0.59) multidentate 2-hydroxyisophthalamide (IAM) terbium chelate (Petoud et al., J Am Chem Soc 2003, 125, 13324). TMP is covalently linked to a proprietary analog of IAM complex (Lumi4) that has similar brightness and a luminescence lifetime (ca. 2.7 ms) that remains unchanged upon conjugation to proteins (see WO 2008/063721, incorporated herein by reference in its entirety). Each of the TMP-LCs that were prepared as described below exhibited characteristic terbium luminescence when complexed with the metal.

In various embodiments, protein-targeted LC probes are delivered to the interior of living cells via several different methods: 1) passive diffusion from culture medium through the cell membrane; 2) reversible physical disruption of the cell membrane; 3) pinocytosis of culture medium containing LC probes followed by osmotic lysis of pinosomes; 4) covalent attachment to Cell-Penetrating Peptides, or other cell-penetrating carrier molecules; and/or 5) microinjection into the cytosol.

Passive diffusion. Small molecules can enter cells via passive (energy-independent) diffusion through the lipid bilayer of a cell membrane or through protein pores in the lipid bilayer. Conjugates of protein-binding ligands to LCs are often relatively large molecules (molecular weight greater than 1000 dalton) and often are charged. These characteristics can preclude passive diffusion into cells from culture medium. Thus, in one aspect, LC probes are synthetically modified to enhance diffusion by altering the overall charge of the molecule, or by introducing amphiphilic moieties such as polyethylene glycol. In some aspects, the charge is altered by adding negatively charged functional groups to the molecule (eg., COO—) to balance the positive charge of the lanthanide cations, with the overall goal being a molecule with a net charge of zero. In other aspects, one or more polyethylene oxide functionalities are covalently coupled to the LC probe to increase amphiphilicity. LC probes that are not permeable to cell membranes are, in other embodiments nevertheless introduced into cells via other means.

Physical disruption of cell membranes. Physical methods such as scrape loading or bead loading are commonly used to load macromolecules or other membrane-impermeable molecules into living cells, and these methods could be used to deliver LCs to intracellularly expressed fusion proteins. [P. L. McNeil, E. Warder, J. Cell Sci. 1987, 88, 669] Here, in one aspect, cells are exposed culture medium containing LC probes, and the cell membranes are temporarily and reversibly disrupted, allowing probe molecules to enter the cytosol. Physical perturbation of adherent cell layers by scratching or scraping the substrate upon which cells are adhered (i.e., scrape loading) introduces temporary lesions in cells at the periphery of the scratch or scrape. When scraping or scratching is applied in the presence of LCs, the LC molecules will enter cells through cell membrane lesions. Agitation of adherent cells in the presence of small glass beads (less than 1 mm diameter) and in the presence of LCs (i.e., bead loading) will introduce temporary lesions through which LCs may enter the cells.

Pinocytosis/Osmotic lysis. Living cells exposed to hypertonic culture medium containing LC probes will undergo pinocytosis. Hypertonic culture medium contains sucrose (0.1 M to 1 M) and polyethylene glycol (1% to 50% by weight) and LC probes (less than or equal to 10 M). Subsequent exposure to hypotonic medium (for example and without limitation, culture medium diluted greater than 25% with water) will cause pinosomes to burst, delivering LC probes to the cytosol of the living cells. [Okada et al., Cell 1982, 29, 33-41].

Cell-penetrating peptides. Cell-penetrating peptides (or cell-permeable peptides, CPPs) have been used to deliver fluorophores, nucleotides, antibodies, and nanoparticles to the interior of living cells (Futaki, Int. J. Pharma. 2002, 245, 1-7; Drin et al., J. Biol. Chem. 2003, 278, 31192-31201; Säälik et al., Bioconjugate Chem. 2004, 15, 1246-1253; Maiolo et al., Biochimica et Biophysica Acta 2005, 1712, 161-172; Fischer et al., ChemBioChem 2005, 6, 2126-2142; Rhee et al., J. Biol. Chem. 2006, 281, 1233-1240; Rappoport, Biochem. J. 2008, 412, 415-423; Wender et al., Adv. Drug. Deliv. Rev. 2007, 60, 452-472). CPPs are composed of various amino acids and often contain repetitive units of arginine. When covalently coupled to otherwise impermeant macromolecules or other “cargo,” the CPP-cargo conjugates usually enter cells via an endocytic pathway, resulting in delivery of cargo to endosomes, with some cargo entering the cytosol or nucleus. In the assemblies, the CPPs represent the penetrating unit that allows the transduction in the cell carrying the luminescent cargo. Examples of CPPs include, for example and without limitation, oligo-arginine, TAT-peptide, and rabies virus glycoprotein. Differential uptake of these transporters has been reported for various cells (Wender et al., Proc. Natl. Acad. Sci. 2000, 97, 13003-13008; Wang et al., J. Clin. Invest. 2002, 109, 1463-1470).

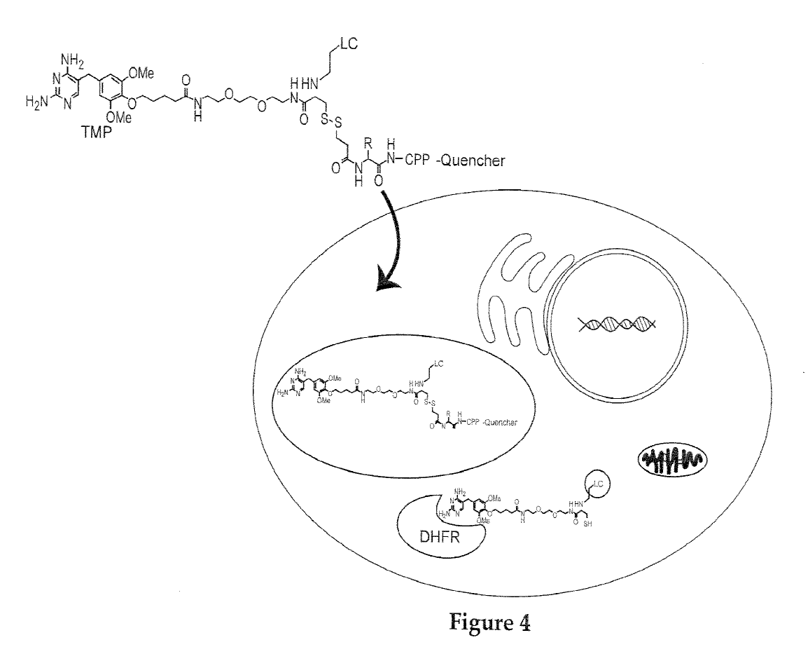

Protein-targeted LCs can be covalently linked to either the N-terminus or C-terminus of a CPP (FIG. 4). Upon addition to culture medium, the CPP-linked LC probe is transported into the interior of the cell. In one embodiment (shown), the LC is coupled to the CPP via a reducible disulfide bond. In this instance, any CPP-LC conjugate that escapes endosomes will be exposed to the reducing environment of the cytosol, resulting in cleavage of the disulfide bond. A quencher molecule can also be linked to the CPP, which serves to reduce luminescence from the LC until it is freed from the CPP by reductive cleavage of the disulfide bond. A quencher is defined as any non-luminescent molecule that absorbs light in the wavelength range corresponding to LC emission. When a quencher is within close proximity to the LC, it effectively reduces or eliminates LC luminescence.

### Microscopic Detection of LRET in Living Cells

The invention also provides microscopic detection of a recombinant fusion protein labeled with an extracellularly administered LC in living cells. Time-resolved epi-fluorescence microscopy is used to detect terbium complex-labeled eDHFR in living mammalian cells. Studies using time-resolved microscopy allow for detection of lanthanide-labeled proteins with extremely high signal-to-background ratio because it is possible to temporally discriminate against short-lifetime scattering and cellular autofluorescence. These studies allow one to chemically optimize LC structure to enhance cell permeability and target specificity, enabling, for example, the use of LC protein labels as long-lifetime luminescent donors in resonant energy transfer studies of protein-protein interactions in vivo.

A wide-field, epi-fluorescence microscope is easily adapted for time-resolved imaging of LCs in living cells, and various embodiments have been reported (Connally, et al. J Biomed Opt 9, 725-734 (2004)). In some embodiments, the living cell is a mammalian cells. In one aspect, a pulsed UV source (e.g., Xenon flashlamp) and an interline transfer, charge coupled device (CCD) detector is integrated with the microscope. The pulsed source and CCD is synchronized so that the CCD is “off” during the excitation pulse (duration=ca. 1-5 μsec). Then, the CCD is turned “on” after a suitable delay (10-50 sec), during which time any short-lifetime (ca. 1-10 nsec) autofluorescence and scattering background is diminished. During the CCD exposure time (ca. 1-2 msec), luminescence from the LC probes is detected. Each light-sensitive pixel in the interline CCD sensor has an associated memory pixel (masked to block light). Before exposure, the CCD is initialized, and then the photoinduced charge (generated during the previous exposure) is shifted to the memory pixels. This procedure is optionally repeated for multiple exposures and then the stored image is transferred to the computer. By capturing multiple images with varying exposure delays and/or exposure times, it is possible to generate an image of the LC donor lifetime (or the sensitized acceptor lifetime) within the cells.

### High Throughput, Cell-Based Detection of Protein-Protein Interactions Using Protein-Targeted LCS

While microscopic analysis of LRET allows for the dynamic, quantitative study of protein-protein interactions in living cells in real-time, it is also beneficial to detect the formation or (especially) inhibition of protein-protein interactions in cells in a high-throughput manner (Inglese et al. Nat. Chem. Biol. 3, 466-479, (2007)). High throughput, time-resolved fluorescence (HTRF) assays are well established techniques that combine a FRET process with time-resolved detection to probe biomolecular interactions in vitro (Trinquet et al. Mol. BioSyst. 2, 380-387 (2006)). Here again, a long-lifetime, luminescent lanthanide donor is bound to one biomolecule, while a short-lifetime acceptor fluorophore is bound to another biomolecule. Any interaction between the two, labeled biomolecules is detected as a change in donor intensity and/or lifetime, or as an increase in sensitized acceptor intensity/lifetime. By analyzing both changes in donor/acceptor intensity ratio as well as changes in donor or acceptor lifetime, it is possible to eliminate most background interference.

HTRF methods have not yet been used for cell-based (in vivo) screens of protein-protein interactions due to lack of suitable protein-targeted, long-lifetime probes. Protein-targeted LC's makes it possible to analyze protein-protein interactions in living cells. This use of protein-targeted LC's is a substantial advantage because it eliminates the need for expressing and purifying proteins. Also, the degree of interaction within the cell may be different than that observed in vitro. In one particular example, and without limitation, cells expressing two interacting proteins, fused to eDHFR and GFP respectively, are grown in multiwell plates (e.g., 96-well, 384-well). Upon addition of cell-permeable TMP-LC conjugate to cells, LRET between TMP-LC bound to eDHFR and GFP occurs if the fusion proteins interacted in the cells. The interaction is detected at high signal-to-background ratio by measuring the sensitized emission of GFP upon excitation of TMP-LC in a time-resolved manner using a fluorescent plate reader. In one aspect, a library of drug-like compounds is added to the cells. In wells where a drug inhibited the protein-protein interaction, a change (decrease) in the sensitized GFP emission is detected. Thus, TMP-LC probes enables high-throughput, cell-based screens of compounds that inhibit protein-protein interactions.

### Target Molecules

The general labeling strategy entails genetically fusing a target molecule to a receptor protein, protein domain or peptide sequence (FIG. 1A). The term “target molecule” is defined herein as a molecule of interest. The interest may be due to a role that the target molecule plays in an important biological process, such as cell proliferation, carcinogenesis, migration, metastasis, differentiation, or apoptosis. Alternatively, the interest may derive from a desire to study the expression and/or function of the molecule. For example, the target molecule may interact with other molecules in a biochemical pathway that researchers are attempting to define. The target molecule may in addition or alternatively be of interest because its expression and/or function may be altered in the context of an assay system used to identify agents that alter the expression and/or function of the target molecule. For example, an assay is used to identify agents, useful in medicine or industry, that modulate the expression of a target molecule, that alter the subcellular localization of a target molecule, or that increase or decrease activity of a target molecule. The cell in which the target molecule resides is referred to herein as the “host cell.”

The target molecule is any naturally occurring or synthetic molecule. For example, it is in one aspect a protein (including but not limited to a glycoprotein, a phosphoprotein, or a lipoprotein, with or without enzymatic activity, and in another aspect is an antibody portion.

In various aspect, the target molecule may reside at the cell surface and may have at least a portion in contact with the extracellular space. Alternatively, the target molecule may be intracellular. The target molecule may, in non-limiting embodiments, have a portion which is, in vivo, embedded in a membrane.

In one aspect, a linker between the target protein and the detector protein in the fusion protein is contemplated. When present the linker does not substantially functionally alter either component, so that neither the normal biological activity of the target molecule nor the affinity of the detector protein for its ligand are substantially disrupted.

### Detector Molecule/Ligand Pairs

A “detector molecule,” as defined herein, is a molecule that can be fused to a target molecule and maintain the ability to associate with a ligand componet of an LC. The fusion of ligand to detector molecule is always structural although it may be direct or indirect. The ligand is in one aspect capable of binding different detector molecules, and the detector molecule is, in certain aspect capable of binding a number of different ligands. As an example, and without limitation, a glucocorticoid receptor molecule, as a detector protein, can bind to a variety of ligand steroid molecules, including agonists such as dexamethasone as well as antagonists such as RU-486; in each case binding is specific but different ligands can bind to the same receptor.

The detector molecule is a protein. Protein subclasses suitable as detector proteins include but are not limited to enzymes, DNA binding proteins, receptors, antibodies fragments and cytostructural proteins.

In certain aspects, the detector molecule is modified to facilitate its intracellular localization. For example and without limitation, where the detector molecule is a protein, it is modified to include a membrane targeting signal. Such membrane targeting signal sequences are known to those of skill in the art.

The choice of a detector molecule/ligand pair to label a target molecule is influenced by a variety of factors. First, because the target molecule is fused to the detector molecule, the functionality of both should not be substantially affected. Second, the target molecule/detector molecule should be accessible to ligand; for example, where the target molecule/detector molecule reside in the cytoplasm the ligand is able to penetrate the cytoplasmic membrane; where the target molecule/detector molecule reside in the endoplasmic reticulum the ligand is able to enter the endoplasmic reticulum, etc. Third, where there is a particular label that is desirable to attach to the ligand (for example and without limitation, a fluorescent compound to be used as an adjunct to GFP), ability of the labeled ligand to bind to the detector molecule should not be substantially decreased by, for example, steric hindrance or electrostatic interactions. Fourth, depending on the duration and nature of labeling of molecular target/detector molecule to labeled ligand, the potential effect of the labeled ligand on the host cell should be considered; for example, the labeled ligand may be toxic to the host cell at certain concentrations and after a certain period of time. Fifth, in a related concern, biological activity of the detector molecule may perturb the function or viability of the host cell, particularly if a threshold amount of the molecule is exceeded, so that if the molecular target is to be produced at high concentrations, a detector molecule should be chosen which can be present at such concentrations without being toxic. Sixth, if the detector molecule has an endogenous counterpart in the host cell, it may be desirable to reduce signal from binding of labeled ligand to endogenous molecule, for example, by using a ligand and/or detector molecule with distinctive structure(s) so that binding to detector molecule is favored. Other factors to be considered would be apparent to the person skilled in the art.

### Ligands

The term “ligand”, as defined herein, encompasses, but is not limited to, molecules that bind to receptors (e.g. a steroid compound binds to a glucocorticoid receptor), molecules that bind to specific targets (e.g., TMP binding to DHFR), cofactors (e.g., heme for binding to hemoglobin or a subunit thereof), functional inhibitors, and substrates (e.g., clavulinate is a suicide substrate for beta-lactamases in penicillin-resistant bacteria). As contemplated herein, a ligand for use in the compositions and methods of the present disclosure is, in one aspect, TMP. Referring to a molecule as a “ligand” relative to its binding partner does not mean that it is smaller than its binding partner, but to facilitate host cell entry and transport, smaller molecules are contemplated. In one non-limiting embodiment, the ligand is a small molecule having a molecular size of 500-2000 daltons.

Any suitable label known in the art is contemplated for use. Non-limiting examples of fluorescent labels include fluorescein, tetramethylrhodamine, Amplex-Red, coumarin, rose bengal, Texas red and Bodipy® fluorophores. Non-limiting examples of chromogenic labels include BCIP (5-bromo-4-chloro-3-indoyl phosphate), a substrate of alkaline phosphatase, which is used in conjunction with nitro blue tetrazolium and X-Gal (5-bromo-4-chloro-3-indoyl B-D galactopyranoside), a substrate of β-Galactosidase.

Regarding the effects of ligand or detector molecule on the host cell and/or the reduction of background signal resulting from binding of labeled ligand to an endogenous counterpart of the detector molecule, the present disclosure contemplates use of natural or synthetic variants of ligands and detector molecules endogenous to a host cell which avoid these problems. The term “variant” as used herein considers the detector molecule or its ligand relative to an endogenous counterpart in the host cell; a naturally-occurring E. coli DHFR detector protein in a mammalian cell would be considered a variant. A mutant of the endogenous mammalian DHFR of the host cell or a mutant of the E. coli DHFR would also be considered to be “variants”.

To avoid ligand toxicity, in certain aspects, the ligand is selected or is structurally modified to disfavor its binding to any endogenous counterpart of the detector molecule. In other aspects, the ligand is selected or modified to have a high affinity for the detector molecule and a low affinity for its endogenous counterpart, where binding to the detector molecule has little if any biological effect. In one non-limiting example where the detector protein is an enzyme, the labeled ligand is a suicide substrate for the detector protein without substantially binding to and/or without inactivating significant amounts of a corresponding endogenous enzyme.

In particular non-limiting embodiments of the invention, both a ligand and a detector molecule are selected or modified to improve the specificity of binding and, in certain instances, to avoid undesirable activities of the ligand and/or detector molecule. A number of detector molecules originating in organisms evolutionarily distant from the host cell and naturally occurring detector molecule variants are available, as may be ligands which selectively bind such detector molecules. Further, methods of redesigning interfaces between ligands and their binding partners are known in the art (see, for example, Clackson et al., Proc. Natl. Acad. Sci. U.S.A. 95:10437-10442 (1998); Clackson, Curr. Opin. Structural Biol. 8:451-458 (1998)).

In additional embodiments of the invention, the ligand is structurally modified to improve its access to or retention in a desired cellular location. For example, the ligand's ability to cross a cell membrane is improved by attaching a lipophilic portion (for example, via an ester linkage that could be cleaved inside the cell) or by “piggy-backing” the ligand on a second molecule. Ligand (modified or unmodified) is in certain aspects incorporated into a microparticle which is taken up by a cell via a clathrin-coated vesicle or other uptake mechanism, uptake may be facilitated by a permeabilizing agent such as dimethylsulfoxide, or ligand export mechanisms are inhibited.

The following are non-limiting examples of detector protein/ligand pairs that are used according to the invention: DHFR/antifolate; glucocoritcoid receptor/steroid (or glucocorticoid receptor/agonist or glucocorticoid receptor/antagonist); TET-repressor/tetracycline; penicillin binding proteins/penicillin or cephalosporin (fluorescently labeled penicillins are commercially available, such as BOCILLIN FL and BOCILLIN 650/665 (Molecular Probes, Inc., Oreg.)); acetylcholinesterase/acetylcholine (fluorescently labeled acetylcholine is commercially available, such as Amplex Red acetylcholine (Molecular Probes Inc., Oregon)); carboxypeptidase A/MTX; cyclophilin prolyl isomerase/cyclosporin; FK506-binding protein (FKBP)/FK506 and rapamycin; beta-lactamase/clavulinate; DNA binding site/DNA binding protein; and hemoglobin/heme. For cyclophilin prolyl isomerase/cyclosporin and FK506-binding protein (FKBP)/FK506 and rapamycin, strategies for redesigning ligand/protein interfaces and modified structures are set forth in Clackson, Curr. Opin. Struct. Biol. 8:451-458 (1998).

In certain non-limiting embodiments of the invention, the detector protein is DHFR. DHFR is an enzyme involved in de novo synthesis of purines and pyrimidines, the building blocks of nucleic acids. DHFR adds two hydrogens to dihydrofolic acid, producing tetrahydrofolic acid. Methotrexate (“MTX”) tightly binds to the active site of the enzyme, thereby inhibiting nucleotide biosynthesis. The anti-proliferative toxic effect of MTX is particularly apparent in rapidly dividing cells, making MTX useful as a chemotherapeutic agent. Regarding the toxicity of MTX, DNA damage has been observed to occur in human cells at an extracellular concentration of 10 micromolar, the toxicity in human leukocytes observed at 2 micromolar.

The present invention provides for the use of DHFR as a detector protein which is directly or indirectly linked to a molecular target. In one non-limiting embodiment of the invention, DHFR (or a portion thereof comprising the active site) and a protein target are both comprised in a fusion protein. DHFR or a portion thereof is positioned at the amino or carboxy-terminus of the target protein; one orientation or the other may preserve the functional characteristics of the proteins. The fusion protein comprises at least the active site of DHFR, to allow for binding of labeled MTX or an MTX analog. DHFR is, in certain aspects, of human or non-human origin.

MTX is optionally chemically modified without disrupting receptor binding by adding modifications at the γ-carboxylate position (Benkovic et al., Science 239:1105-1110 (1988); Bolin et al., J. Biol. Chem. 257:13650-13662 (1982)). Accordingly, in one aspect, MTX is chemically linked to a wide variety of fluorophores or chromophores. Indeed, a number of methotrexate-conjugated fluorophores are commercially available from Molecular Probes (Eugene, Oreg.), including without limitation fluorescein-methotrexate, Texas Red™-methotrexate, BODIPY™-methotrexate, and AlexaFluor™-methotrexate. References describing fluorescently labeled MTX include Gapski et al., J. Med. Chem. 18:526-528 (1975); Fan et al., Biochem. 30(18):4573-4580 (1991) and Rosowsky et al., J. Biol. Chem. 257(23):14162-14167 (1982).

Several approaches are used in various aspects in order to avoid the toxic effects of MTX on host cells. The specific DHFR detector protein are in one aspect selected such that a labeled ligand favors binding to the detector protein rather than endogenous DHFR.

In one series of embodiments, a naturally occurring DHFR from a species other than that of the intended host cells is used which binds to substrates that do not have high affinity for endogenous DHFR. For example, DHFR from E. coli is used as a detector protein, and labeled trimethoprim, which favors binding to the bacterial DHFR over its mammalian counterpart, is used as ligand. Trimethoprim (2,4-Diamino-5-(3,4,5-trimethoxybenzyl) pyrimidine, TMP) binds tightly to the E. coli form of Dihydrofolate Reductase (KI=ca. 1 nm). However, trimethoprim's affinity for mammalian forms of DHFR is over 1000-fold lower (KI=ca. 3700 nm; Baccanari et al., Biochemistry 21: 5068-5075 (1982)). This selectivity for the E. coli form of DHFR is exploited for the labeling and detecting of proteins in mammalian cells. Trimethoprim that has been functionalized with a fluorescent or otherwise detectable label is in one aspect used to selectively bind to an E. coli DHFR fusion protein expressed in mammalian cells without binding appreciably to endogenous DHFR. This is important to minimize background. Thus, trimethoprim and E. coli DHFR comprise an orthogonal ligand-receptor pair for the purposes of labeling and detecting fusion proteins in all mammalian cell types.

In specific, non-limiting embodiments of the invention, labeled TMP is prepared which is substituted at the 4′ position. The 4′-substituted TMP retains nanomolar affinity for E. coli DHFR as well as selectivity over mammalian forms of DHFR if the substituent is attached via an alkyl linker with a chain length longer than three carbons (Roth et al., J. Med. Chem. 24: 933-941 (1981); Kuyper et al., J. Med. Chem. 28: 303-311 (1985)). 4′-alkylamino-substituted TMP is in one aspect prepared as follows. TMP is selectively demethylated at the 4′ position according to the method of Brossi et al., J. Med. Chem. 14: 58-59 (1971). The resulting phenol compound is alkylated via reaction with a bromo-alkanoate ester (Kuyper et. al., J. Med. Chem. 28: 303-311 (1985)). The ester is then hydrolyzed and reacted with a diamino alkane of desired length using a carbodiimide, or other peptide coupling reagent. The product, a 4′-alkylamino-substituted TMP is then linked to commercially available, amine-reactive fluorescent molecules (e.g., Texas Red®-X succimidyl ester, fluorescein succimidyl ester, malachite green isothiocyanate; all available from Molecular Probes, Eugene, Oreg.).

In additional embodiments of the invention, a DHFR is structurally modified to alter its substrate binding characteristics and thereby confer desired selectivity and/or binding affinity.

Further, various mutations are known which result in MTX resistance. These mutations include, but are not limited to, substitutions in human DHFR at position 22 (leucine) to arginine or phenylalanine and at position 31 (phenylalanine) to serine or tryptophan (Banerjee et al., Gene 139(2): 269-274 (1994); Morris and McIvor, Biochem. Biopharmacol. 47(7):1207-1220 (1994); Simonsend and Levinson, Proc. Natl. Acad. Sci. U.S.A. 80(9):2495-2499 (1983)). MTX is in one aspect modified to bind to the active site in DHFR natural or synthetic variants that do not bind unmodified MTX with sufficient affinity.

Where DHFR is the detector protein, the extracellular concentration of ligand is in one aspet between 1 nanomolar and 100 micromolar. In certain non-limiting embodiments, the concentration is between 0.1 and 10 micromolar. For uses in which a host cell is exposed to labeled ligand for a prolonged period of time, a dose is used which will not result in significant toxicity; for example but not by way of limitation, less than 10 micromolar or less than 2 micromolar.

### Methods of Use

In certain embodiments, the present invention provides methods of localizing and following a target protein fused to a detector protein.

For example, in a set of non-limiting embodiments, a nucleic acid construct is produced which encodes a first protein target fused (at its amino terminus or carboxy terminus) to a first detector protein. A second construct is produced which encodes a second protein target fused (at its amino terminus or carboxy terminus) to a second detector protein. The fusion constructs are in one aspect operatively linked to a promoter which is, depending on experimental design, the naturally occurring promoter of the target protein (a “target homologous promoter”) or a heterologous promoter. The promoter/fusion constructs are introduced into a host cell using standard techniques (for example and without limitation, via a viral vector, transformation or transfection). The host cell is cultured under standard conditions known in the art. A LC is then introduced into the host cell. The LC is in one aspect introduced into the host cell during the culture period or in one or more separate step(s), including prior to the start of culture or after culture. In non-limiting embodiments of the invention, the host cell is exposed to a LC at a non-toxic concentration and/or for a period of time which will minimize toxicity. The LC associates with the first detector protein. Similarly, the first protein target associates with the second protein target (which is fused to the second detector protein). The resulting proximity of the LC with the second detector protein results in FRET, which is detected using standard techniques. For example, where the second detector protein is a fluorescent label, the location of LC bound to the first detector protein (and hence the location of target protein) is in one aspect detected using fluorescence microscopy.

While the present invention has been described in terms of various embodiments and examples, it is understood that variations and improvements will occur to those skilled in the art. Therefore, only such limitations as appear in the claims should be placed on the invention.

### EXAMPLES

### Example 1

### Synthesis of trimethoprim-lanthanide complexes (TMP-LCs)

Precursors. Triethylenetetraminehexaacetic acid dianhydride (6b) was synthesized from triethylenetetramine-N,N,N′,N″,N′″,N′″-hexaacetic acid as previously described (Zitha-Bovens et al., Helvetica Chimica Acta, 88, 618 (2005)). Confirmed by 1H NMR (400 MHz, D₂O) δ 3.20-3.24 (m, 8H), 3.37 (m, 4H), 3.65 (s, 4H), 3.79 (s, 8H) and melting point 171-172° C. Trimethoprim (TMP) was converted to a boc-protected amine derivative (5, Scheme 6) as described below.

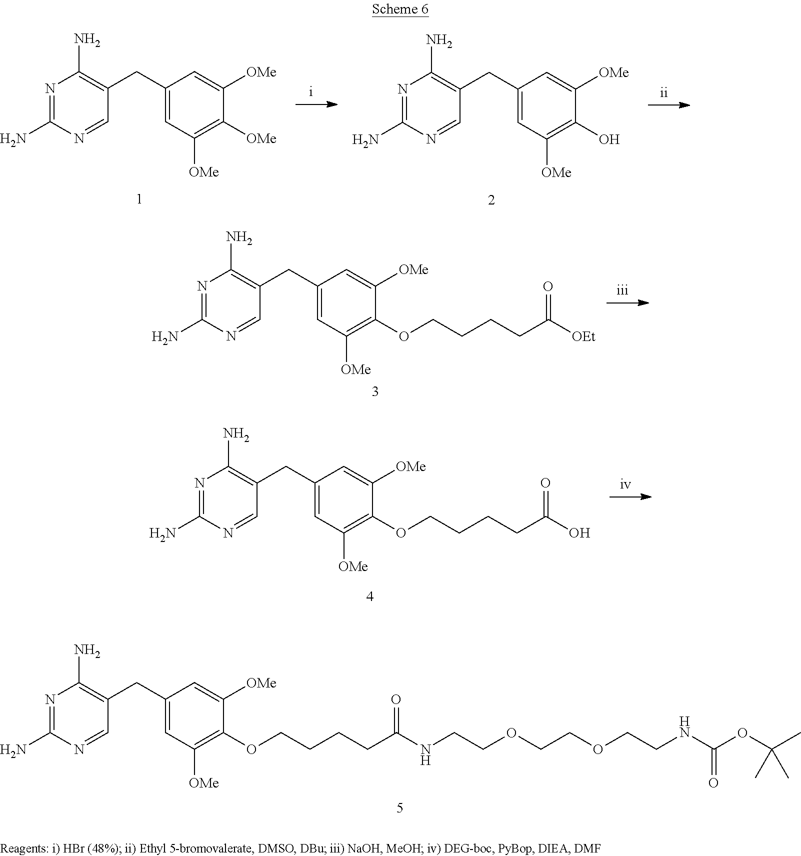

2: Trimethoprim, 1, (5.00 g, MW: 290) was added to a round bottom flask containing HBr (60 mL, 48%) refluxing at 95° C. The solution was stirred under air for 20 min and the temperature was maintained with an internal temperature probe. The solution was then partially neutralized with NaOH (15 mL, 50% w/w). Stirring was stopped and the solution was allowed to cool to room temperature and then placed in 4° C. refrigerator overnight resulting in beige needle-like crystals. The crystals were filtered from solution and recrystallized by dissolving in a minimal amount of cold H₂O, which was then neutralized to pH 7 with NH₄OH. The solution was chilled at 4° C. and the resulting pure crystals were filtered to yield 2 (2.75 g, 10.0 mmol, 58%) ¹H NMR (400 MHz, [D4]CH₃OH, 25° C.): δ=7.51 (s, 1H, ArH), 6.63 (s, 2H, ArH), 3.85 (s, 6H, ArOCH3), 3.65 (ArCH₂Ar).

3: Molecule 2 (2.75 g, MW: 276.29, 10.0 mmol) was dissolved in DMSO (30 mL) and DBU (11 mmol) was added. After dissolving, the solution turned a deep red and ethyl 5-bromovalerate (11 mmol) was added. The solution was stirred for 30 min at room temperature, becoming green. Water (100 ml) was added and the product was extracted with EtOAc (4×100 ml). The combined EtOAc solution was washed with water (200 ml), dried over MgSO₄, and evaporated. The brown oil was subjected to column chromatography on silica gel, elution with 10% CH₃OH in CH₂Cl₂. Fractions containing the desired product were pooled and concentrated to dryness yielding 3 (1.09 g, 2.70 mmol, 27%). ¹H NMR (400 MHz, [D₄]CH₃OH, 25° C.): δ=7.54 (s, 1H, ArH), 6.57 (s, 2H, ArH), 4.12 (m, 2H, COOCH₂), 3.91 (m, 2H, ArOCH₂), 3.77 (s, 6H, ArOCH₃), 3.57 (s, 2H, ArCH₂Ar), 2.48 (t, 2H, CH₂COO), 1.65-1.85 (m, 4H, CH₂CH₂), 1.26 (m, 2H, CH₃).

4: Molecule 3 (1.09 g, MW: 404.46, 2.7 mmol,) was dissolved in methanol (15 mL). To the solution was added NaOH in water (50% w/w, 8.1 mmol). The solution was then stirred under air for 1 hour and then titrated to pH 4 with HCl (1 N). The resulting beige crystals were filtered from solution, washed with brine and water, yielding 4 (681 mg, 1.8 mmol, 67%). ¹H NMR (400 MHz, [D₄]CH₂SOCH₂, 25° C.): δ=7.42 (s, 1H, ArH), 6.59 (s, 2H, ArH), 3.81 (m, 2H, ArOCH₂), 3.77 (s, 6H, ArOCH₃), 3.57 (s, 2H, ArCH₂Ar), 2.25 (t, 2H, CH₂COO), 1.65-1.85 (m, 4H, CH₂CH₂).

5: Molecule 4 (681 mg, MW: 376.41 1.8 mmol) and PyBOP (1.8 g, 3.6 mmol, 2 eq.) were added to a round-bottom flask and placed under vacuum. After ca. 1 hour, DMF (20 mL) and tert-Butyl-2-(2-(2-aminoethoxy)ethoxy)ethylcarbamate (DEG-Boc, 536 mg, 2.16 mmol, 1.2 eq.) were added to the flask under N2. Diisopropyl ethylamine (DIEA, 10 eq.) was added, and the reaction was stirred overnight at room temperature. The solvent was removed by rotory evaporation and mixture of crude products were isolated by flash chromatography (CH₃OH:CH₂Cl₂ 1:19 over SiO₂), yielding 5 (295 mg, 0.49 mmol, 27%). ¹H NMR (400 MHz, [D₄]CH₃OH, 25° C.): δ=7.29 (s, 1H, ArH), 6.55 (s, 2H, ArH), 3.91 (s, 2H, ArOCH₂), 3.79 (s, 6H, ArOCH₃), 3.73-3.69 (mult, 14H, ArCH₂Ar+DEG linker H) 2.30 (t, 2H, CH₂CONH), 1.70-1.80 (m, 4H, CH₂CH₂), 1.42 (s, 12H, O(CH₃)₃).

tert-Butyl-2-(2-(2-aminoethoxy)ethoxy)ethylcarbamate (DEG-Boc): 2-(2-(2-Aminoethoxy) ethoxy) ethanamine (6 gm, 40.54 mmol) was dissolved in a solution of triethyl amine methanol (10% TEA in CH₃OH, 130 mL). A solution of di-tert-butyl dicarbonate (2.95 gm, 13.53 mmol) in methanol (10 mL) was added to this mixture with vigorous stirring. The mixture was refluxed for 2 h and left to stir at room temperature overnight. The excess methanol and TEA were removed in vacuo to yield an oily residue that was dissolved in dichloromethane and washed with 10% aqueous sodium carbonate. The organic layer was separated, dried over MgSO₄ and filtered, and the solvent was removed in vacuo. The oily residue was filtered through a bed of silica.

Compounds 7a (TMP-cDTPA) and 7b (TMP-cTTHA). The Boc group was removed from 5 by stirring in TFA in CH₂Cl₂ (50% v/v) for 5 hours. Milligram-scale quantities of TMP-polyaminocarboxylates were prepared in a 3-component reaction with the appropriate anhydride, carbostyril 124 (cs124), and deprotected 5 (Scheme 7). 6a or 6b were dissolved in DMF (100 mM) and triethylamine (20 equiv.). A mixture of cs124 (1.0 equiv., 100 mM) and 5 (1.0 equiv., 100 mM) dissolved in an equal volume of DMF was added while stirring under N2 atmosphere. The reaction proceeded for 18 hours at room temperature, and was then quenched by addition of 2 volumes distilled water. Products were purified by high performance liquid chromatography (HPLC) with a C18 column (GraceVydac, cat. no. 218TP54, 5 μm, 4.6 mm i.d.×250 mm). A 20-min linear gradient, from 5% to 35% solvent B (solvent A, 0.1 M triethylammonium acetate (pH 6.5) plus 5% CH₃CN; solvent B, acetonitrile) was used. Fractions were pooled, rotovapped to remove acetonitrile, and lyophilized to yield the desired compounds. ESI-MS, TMP-cDTPA (C₄₈H₆₇N₁₁O₁₅): m/z 1038.4 [M+H⁺]; TMP-cTTHA (C₅₂H₇₄N₁₂O₁₇): m/z 1139.6 [M+H⁺].

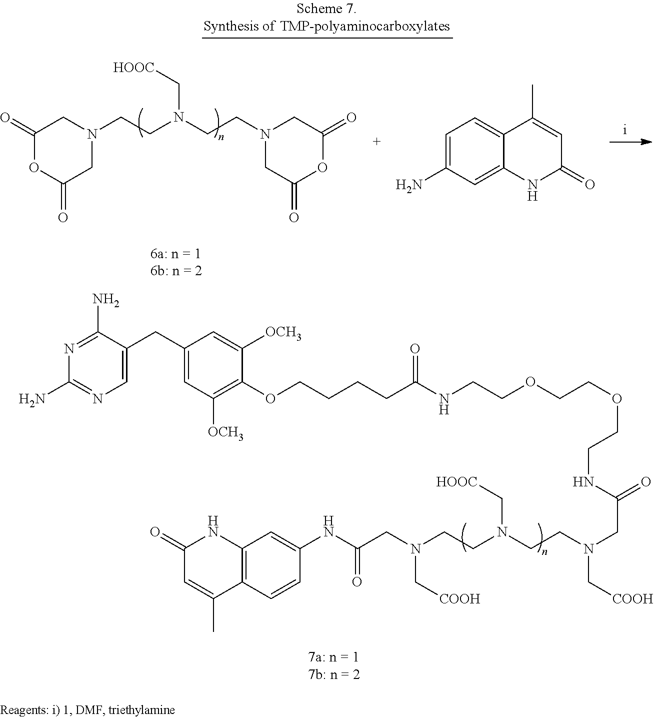

Compound 5 was coupled to Lumi4®-COOH in a single step. Compound 5 (deprotected 0.0025 mmol, 0.01M), N-ethyl-N′-(3-dimethylaminopropyl)carbodiimide (EDC,1.2 equiv.) and 1-hydroxybenzotriazole hydrate (HOBT, 1.2 equiv.) were dissolved in 0.5 mL DMF and 100 uL of triethylamine under nitrogen atmosphere. 240 μL of a 0.005 M solution of the Lumi4®-COOH in DMF was added to the reactants, and the solution was stirred at room temperature for 18 h. Products was purified by HPLC with a C18 column (GraceVydac, cat. no. 218TP54, 5 μm, 4.6 mm i.d.×250 mm). A 20-min linear gradient, from 5% to 35% solvent B (solvent A, 0.1 M triethylammonium acetate (pH 6.5) plus 5% CH₃CN; solvent B, acetonitrile) was used. The fractions containing the desired compound were pooled, rotovapped to remove acetonitrile, and lyophilized to yield the desired compound. ESI-MS⁺ (C₈₅H₁₁₅N₁₉O₂₀): m/z 1722.87 [M+H]⁺.

### Metal Labeling of Compounds

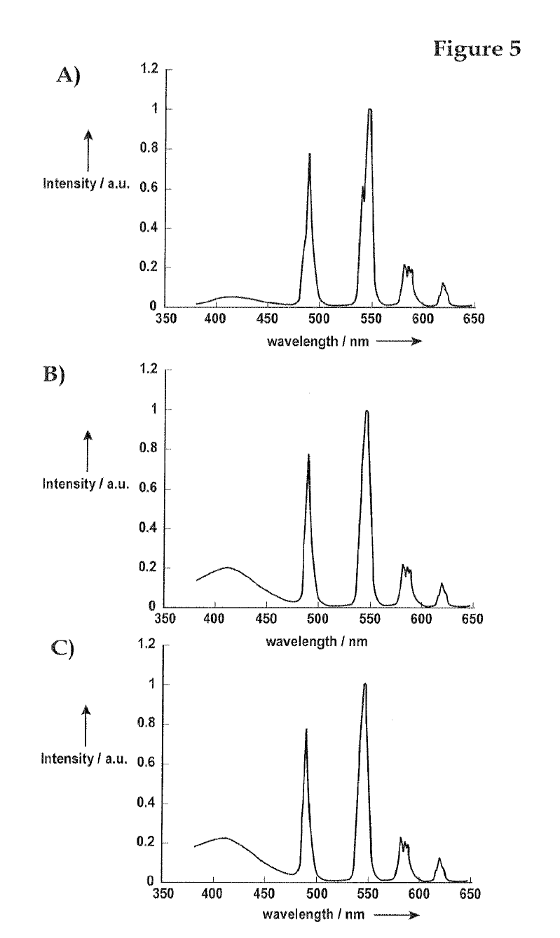

Compound concentrations were calculated using measured absorptions and published extinction coefficients for the fluorophores (cs124, ε=10,500 M⁻¹ cm⁻¹ at λ=343 nm; Lumi4®, E=26,000 M⁻¹ cm⁻¹ at λ=339 nm) (Li et al., J Am Chem Soc 117: 8132 (1995); Petoud et al., J Am Chem Soc 125: 13324 (2003)). Compounds were dissolved in distilled water (5 μM). TbCl₃.6H₂O (0.8 equiv., 5 mM in H₂O) was added, and solution was stirred at room temperature for 15 min. The solutions were titrated with 0.25 mM TbCl₃ and the luminescence intensity measured with a fluorimeter (JobinYvon, Fluoromax 3) until there was no increment in the Tb emission. ESI-MS⁺: (C₄₈H₆₇N₁₁O₁₅Tb), m/z 1197.3 [M+H]⁺; (C₅₂H₇₁N₁₂O₁₇Tb), m/z 1295.44 [M+H]⁺; (C₈₅H₁₁₅N₁₉O₂₀Tb), m/z 1881.3 [M+2H⁺]. Normalized luminescence emission spectra are shown in FIG. 5. Terbium-labeled compounds were lyophilized and stored at −20° C.

### Example 2

### Binding Affinity Assay

The affinity of lanthanide dyes for eDHFR was determined by time resolved FRET. TMP-LCs (20 nM), were titrated in 96-well plates with purified eDHFR-GFP at concentrations ranging from 0.5 nM to 1000 nM in Assay buffer (50 mM K₂HPO₄, KH₂PO₄, 18 mM β-mercaptoethanol, 200 μM NADPH, pH 7.2). Each titration was done in triplicate. Intermolecular FRET from eDHFR-bound TMP-LCs to GFP was detected using a time-resolved fluorescence plate reader (Perkin Elmer, Victor 3V: λₑₓ=340 nm; λₑₘ=520/10 nm; time delay=100 μs; measurement window=1400 μs). Using Kaleidagraph (Synergy Software, PA), the sensitized GFP emission was plotted against protein concentration, and the data were fit to the following equation in order to obtain the dissociation constant:

where F₀ is the FRET of the lanthanide probe with no receptor, F₁₀₀ is the maximum FRET signal with an infinite amount of receptor, [L]_{T} is the total amount of lanthanide probe used, and [P]_{T} is the total amount of protein used.

### Example 3

### Intramolecular TR-FRET

For both in vitro and live cell applications, TMP-TCs must necessarily bind with high affinity to eDHFR fusion proteins. In order to determine whether the TMP-TCs could bind to eDHFR and serve as FRET donors to green fluorescent protein (GFP), a purified eDHFR-GFP fusion protein was titrated against a fixed concentration (20 nM) of the different TMP-TCs.

pRSETb-EGFP-eDHFR. The gene encoding eDHFR was subcloned from plasmid pLL-1 to pRSETb-mTSapphire to generate pRSETb-mTSapphire-eDHFR. A 577 bp BsrGI to EcoRI fragment encoding eDHFR with an N-terminal (Gly-Ser-Gly)₂ linker was prepared by PCR from pLL-1 using the primers 5′-GCA TAC GTC TGT ACA AGG GAT CTG GAG GAT CTG GAA TCA GTC TGA TTG CGG C-3′ (SEQ ID NO: 1) (BsrGI, coding strand) and 5′-GCA TAC GAA TTC TTA CCG CCG CTC CAG AAT C-3′ (SEQ ID NO: 2) (EcoRI, non-coding strand). This fragment was inserted between the BsrGI site and the EcoRI site in pRSETb-mTSapphire to give to pRSETb-mTSapphire-eDHFR. The gene encoding enhanced GFP (EGFP) was amplified by PCR from pEGFP-TUB (Clontech, Inc.) using the primers 5′-GCA TAC GTC GGA TCC CAT GGT GAG CAA GGG CGA GG-3′ (SEQ ID NO: 3) (BamHI, coding strand) and 5′-GCA TAC GTC TCT ACA GCT CGT CCA

$$F = {F}_{0} - ( {F}_{0} - {F}_{100} ) \times \frac{\begin{matrix}( {[ L ]}_{T} + {K}_{D} + {[ P ]}_{T} ) - \\ \sqrt{{( {[ L ]}_{T} + {K}_{D} + {[ P ]}_{T} )}^{2} - ( {{4 [ L ]}_{T} [ P ]}_{T} )}\end{matrix}}{{2 [ L ]}_{T}}$$

TGC-3′ (SEQ ID NO: 4) (BsrGI, non-coding strand) to yield, pRSETb-EGFP-eDHFR. Upon transformation into E. coli, pRSETb-EGFP-eDHFR expressed the protein fusion MRGSHHHHHHGMASMTGGQQMGRDLYDDDDKDP-[EGFP]-GSGGSG-[eDHFR] (SEQ ID NO: 5 (polynucleotide) and SEQ ID NO: 6 (polypeptide)).

EGFP-eDHFR Purification: EGFP-eDHFR was purified from the E. coli strain BL21 DE3 (pLysS) transformed with pRSETb-EGFP-eDHFR. 5 ml of 2 X-TYAC overnight culture was used to inoculate 500 ml of 2X-TYAC (Ampicillin (100 mg/ml), chloramphenicol (34 mg/ml)). The 500 ml culture was grown at 37° C., shaking at 200 rpm, to an OD₆₀₀ of Ca. 0.6, at which time expression of the protein was induced by the addition of IPTG to a final concentration of 1 mM. After growth for another 3 h, the cells were harvested by centrifugation. The pellet was lysed in 30 ml of lysis buffer (50 mM sodium phosphate pH=8, 300 mM NaCl, 10% glycerol, 10 mM imidazole) on ice. After addition of TCEP (to 1 mM), 300 μl PIC, lysozyme (20 mg) and sodium lauryl sarcosine (1%), the lysate was centrifuged (15000 rpm, 15 min 4° C.) and applied to a column containing 1.2 ml of Ni-agarose beads (Qiagen). Following purification, the protein was concentrated (to 30 μM), dialyzed with storage buffer (PBS, pH 7.4), and stored at −80° C.

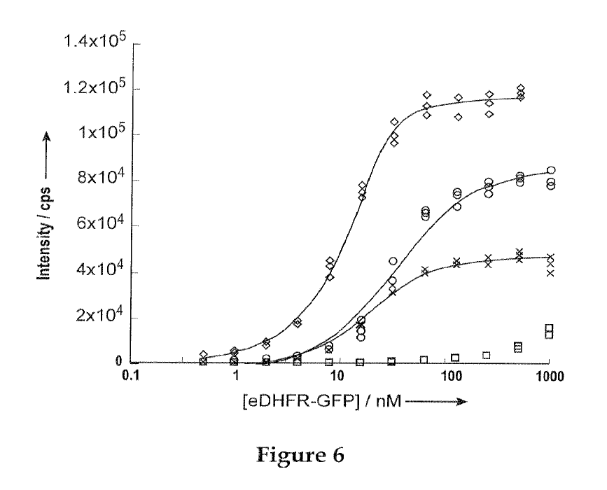

Using a time-resolved fluorescence plate reader, sensitized, long-lifetime (>100 μs) emission of GFP was detected that increased with increasing protein concentration (FIG. 6). Addition of excess TMP abrogated the signal, indicating that intramolecular TR-FRET occurred between the eDHFR-bound conjugates and GFP. The relative intensities of sensitised GFP emission at binding saturation were positively correlated to the reported quantum yields of the complexes. A non-linear, least-squares fit of the data revealed the dissociation constants for binding to eDHFR of TMP-cDTPA, TMP-cTTHA and TMP-Lumi4 to be 9±1.3 nM, 22±3.0 nM and 1.8±0.3 nM, respectively. The measured affinities were lower than a previously reported value for binding of a TMP-fluorescein conjugate to eDHFR (K_{d}=ca. 30 nM) (Miller et al., Nature Methods 2: 255 (2005)) and approach the value of free TMP.

Cell growth, transfection and probing with small molecule: In preparation for transfection, NIH3T3 cells were seeded at 10⁵ cells per well into a 6-well plate. Cells were grown in DMEM medium containing 10% fetal bovine serum and supplemented with 15 mM HEPES, 1% L-glutamine and penicillin (100 Iu/mL)/streptomycin (100 mg/mL) at 37° C. and 5% CO₂. After ca. 18 hrs., adherent cells (ca. 80% confluent) were transfected with 2 μg of the desired plasmid DNA using Lipofectamine-2000™ transfection reagent according to manufacturers instructions. Ca. 6 hrs. after transfection, cells were trypsinized and reseeded into chambered 8-well plates (14,000 cells/well). Ca. 24 h after transfection, indicator-free DMEM containing the desired concentration of TMP-LCs was added to the adherent cells, and the cells were incubated for the indicated duration at 37° C. and 5% CO₂. The cells were then washed 2× with phosphate buffered saline, re-immersed in indicator-free medium, and imaged.

Microscopy: Time resolved fluorescent microscopy of adherent live cells was performed using an epi-fluorescence microscope (Zeiss Axiovert 200M) modified with the following components: 1) Xenon flashlamp (Perkin Elmer FX4401); 2) delay generator (Stanford Research Systems, DG645); 3) a gated image-intensified CCD camera (ICCD, mounted on the side-port of the microscope) and camera controller (Stanford Photonics, Mega-10EX; and 4) a computer running Piper Control software (v2.4.05, Stanford Photonics, Inc.). A 100 W mercury arc lamp was available for continuous wave fluorescence excitation, and a conventional CCD (Zeiss Axiocam MRM) was mounted on the front port of the microscope. Filter cubes containing the appropriate excitation and emission filters and dichroics allowed for wavelength selection (λₑₓ=350/50 nm, λₑₘ=550/20 nm). Time-resolved image acquisition was initiated by a start signal (TTL) from the computer to the delay generator. Separate outputs (TTL) routed from the delay generator to the flashlamp and the ICCD (via the camera controller) relayed a pre-programmed “burst” sequence to trigger the lamp and the intensifier a user-defined number of times. For each acquisition, the signal from multiple excitation/emission events was accumulated on the ICCD sensor and is read out to the image capture card of the computer at the end of the acquisition period. The flashlamp pulse rate (to 1 kHz), the gate delay, the gate width, and the total image acquisition period (66.67 msec-2 sec) were independently variable.

Samples were imaged with a 63X EC Plan Neofluar oil immersion objective (NA=1.25). Images were captured with Piper control software and rendered using NIH Image J and Adobe Photoshop 5.5.

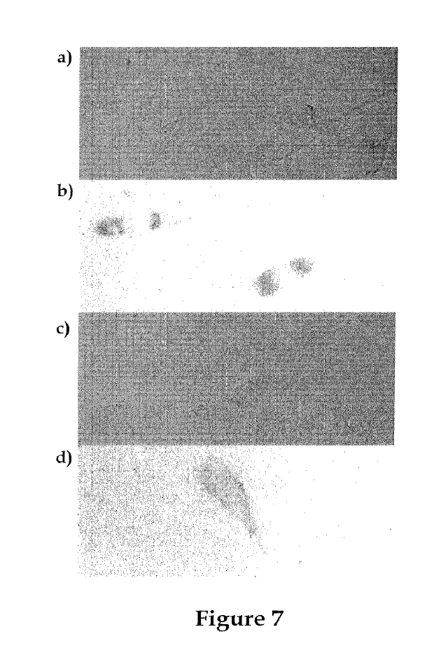

Whether TMP-TCs could be used to label eDHFR fusion proteins in living mammalian cells was then determined. NIH3T3 fibroblast cells were transiently co-transfected with two plasmid DNA vectors; one that expressed plasma membrane-targeted eDHFR and another that expressed nucleus-localized CFP, included as a positive control for transfection. The cells were incubated ca. 20 hours in growth medium containing 100 μM TMP-cTTHA, washed, and imaged using an epi-fluorescence microscope capable of pulsed UV excitation and time-resolved detection. No specific labeling of plasma membrane-localized eDHFR was observed in cells that expressed nucleus-localized CFP (FIG. 7b). Non-specific luminescence was detected in all cells, possibly indicating endocytosis of the compound and trapping in lysosomes. When similar experiments were performed with lower concentrations and/or shorter incubation times, long-lifetime luminescence could not be detected in cells incubated with any of the TMP-TCs.

As a control, cell surface-expressed eDHFR was labeled with a TMP-fluorescein conjugate and imaged using conventional epi-fluorescence microscopy with continuous wave illumination. A heterodimeric conjugate of TMP linked to isobutyryl ester-protected fluorescein (LigandLink™ Fluorescein, Active Motif, Inc., Carlsbad, Calif.) was diluted to a concentration of 10 μM in PBS (pH=9.0), and stored at room temperature for ca. 24 h in order to hydrolyze the ester protecting groups. The deprotected TMP-fluorescein was diluted further to a concentration of 1 μM in growth medium (DMEM). NIH3T3 fibroblasts were co-transfected with the nucleus-localized CFP expression plasmid and pDisplay-eDHFR. Ca. 24 h after transfection, the cells were incubated in DMEM containing 1 μM TMP-Fluorescein for 10 min., washed, and imaged. Faint membrane luminescence was observed only in cells that expressed nucleus-localized CFP. Contrast is comparable to that obtained with time-resolved imaging of cell-surface-localized eDHFR labeled with TMP-Lumi4.

While intracellular labelling of eDHFR with the TMP-LCs was not possible, eDHFR expressed on the cell surface was successfully labeled. NIH3T3 fibroblasts were co-transfected with the nucleus-localized CFP expression plasmid and a vector that expressed eDHFR on the extracellular surface of the plasma membrane (pDisplay-eDHFR). Ca. 24 hours after transfection, the cells were incubated in growth medium containing 1 μM TMP-Lumi4 for 10 min., washed, and imaged. A distinct membrane luminescence was observed only in cells that expressed nucleus-localized CFP when the cells were imaged in time-resolved mode (FIG. 7c-d). The membrane fluorescence could only be detected for approximately 20 min. after washing due to dissociation of the TMP-Lumi4 from eDHFR and diffusion into the medium. A control experiment established that the membrane fluorescence was dependent on the specific labelling of the eDHFR fusion protein with TMP-Lumi4. Pre-incubation of the cells expressing membrane-targeted eDHFR in medium containing 10 μM TMP, followed by incubation in medium containing 1 μM TMP-Lumi4 resulted in no membrane staining. Cell-surface labelling of eDHFR was only detected with TMP-Lumi4, and not with TMP-cDTPA or TMP-cTTHA.

The invention discloses that the high affinity, non-covalent interaction between TMP and eDHFR provides an effective means for imparting terbium luminescence to recombinant fusion proteins. Terbium-complexed TMP-TCs exhibited characteristic luminescence and high affinity for eDHFR, and they proved to be efficient sensitizers of GFP emission in an intramolecular TR-FRET assay. TMP-Lumi4 was particularly effective, binding to eDHFR-GFP with ca. 2 nM affinity and exhibiting>100-fold increase in FRET signal upon binding saturation. As FRET donors to GFP, TMP-TCs could be used to detect interactions between eDHFR and GFP fusion proteins. This would be particularly useful when protein-specific antibodies are unavailable, or in situations where direct conjugation of proteins with terbium complexes is problematic, such as assays of cell lysates. As prepared in the present example, the TMP-TCs are cell-impermeable, and can only be used to label proteins on cell surfaces. However, physical methods such as scrape loading or bead loading that are commonly used to load macromolecules into living cells could conceivably be used to deliver TMP-TCs to intracellularly expressed eDFHR fusion proteins (McNeil et al., J. Cell Sci., 88, 669 (1987)). Alternatively, as described herein, methods employing cell-penetrating peptides (CPPs) may be used to deliver lanthanide probes internally to living cells.

### Example 4

### Time Resolved Fluorometry and Microscopy

CPP-LC conjugates can be screened for total cellular uptake using the methods described by Holm et al. (Holm et al., Nature Protocols 2006, 1, 1001-1005). Briefly, adherent cells are cultured under cell type-dependent conditions in 12-well plates. The cells are incubated with CPP-LC solutions, trypsinized, lysed, and transferred to 96-well plates. A time-resolved fluorescence plate reader is used to quantitatively determine Tb3⁺ luminescence for each sample, and the luminescence is normalized to total protein concentration in the cell lysates. In this way, the CPP-LC conjugates that are most readily taken up by a variety of cell types can be quickly identified.

It is important to determine the sub-cellular distribution of LCs following CPP-mediated delivery. Here again, experimental protocols are based on the method of Holm et al. (Holm et al., Nature Protocols 2006, 1, 1001-1005). Adherent cells are cultured and treated with the most promising CPP conjugates identified in the fluorometric screen. TRM will be used to image the cells and determine 1) the concentration-dependent rate of conjugate transport from culture medium to the cell interior and 2) the sub-cellular distribution of LC.

Since endocytosis is considered a major route for cell entry of CPPs (Holm et al., Nature Protocols 2006, 1, 1001-1005), cell loading experiments are performed with rhodamine-labeled dextran (a marker for endocytosis). This allows for the determination of the extent to which the CPP-LC conjugates co-localize with rhodamine-dextran in endosomes. Furthermore, the viability of the disulfide linker and quenching strategies are determined. Escape from the endosome into the cytosol mediates reduction of the disulfide bond linking LC to the quencher-CPP conjugate, resulting in diffuse luminescence detectable throughout the cell.

In order to ensure that an effective means of loading LCs into cells has been accomplished, the pinocytosis and osmotic lysis method are analyzed for loading LCs into fibroblasts and neuron-like cells. If necessary, bead loading methods can be explored as an alternative strategy for delivery of LC derivatives (McNeil et al., J. Cell Sci. 1987, 88, 669-678). Completion of these experiments will 1) identify which combination of CPP and conjugation strategy most effectively delivers LCs to the cytosol of various cell types; 2) the optimal cell growth, incubation and microscopy conditions for effective delivery and detection; and 3) reagents and delivery conditions for further experiments.

Using methods known in the art, recombinant fusion proteins are labeled with LCs in living cells and detected using TRM. Heterodimeric conjugates of LCs linked to trimethoprim (TMP) bind to Escherichia coli dihydrofolate reductase (eDHFR) fusion proteins with nanomolar affinity. TMP-LC heterodimers are coupled to CPPs and spectroscopically characterized as to their extinction coefficients, fluorescent lifetimes, and quantum yields. Delivery into the cytosol of living cells will also be characterized. Fusions of eDHFR are expressed to sub-cellularly localized proteins or peptide signal sequences and TRM will be used to determine the specificity and stability of labeling in vivo and the degree of any non-specific labeling or compartmentalization.

### Example 5

### Synthesis and characterization of Lumi4®-Tb-TMP-CPP

The LC-TMP-CPP conjugates are assessed for their uptake into the cytosol of living, adherent cells using methods known in the art. Fibroblasts, immortal neuron-like cells, and neurons will be grown on coverslips and treated with the conjugates. Here again, the conjugate(s) and labeling protocols that result in the highest concentration and most uniform distribution of LC-TMP throughout the cytosol are identified, as measured by TRM.

The selective and stable binding of TMP-LC to eDHFR in cells is critical for performing TR-FRET imaging and dynamic analysis of protein distribution. TRM is used to determine three performance parameters for each LC-TMP-CPP that have been identified: 1) the long-term stability of the eDHFR-TMP-LC complex in mammalian cells; 2) the level of unbound, or non-specifically bound probe present in the cells; and 3) the labeling conditions required to achieve maximal signal above background. Mammalian expression vectors that encode fusions of eDHFR under control of the constitutive cyomegalovirus (CMV) promoter are prepared. Fusion of eDHFR with the N-terminal myristoylation/palmitoylation signal sequence of lyn-kinase directs eDHFR to the cytosolic face of the plama membrane (Miller et al., Nat. Methods 2005, 2, 255-7). A vector encoding eDHFR fused to the N-terminus of human cytoplasmic β-actin enables labeling and detection of stress fibers in expressing cells. Non-neuronal cells and neurons are transiently transfected using cationic liposomes or calcium phosphate (Jiang et al., Nature Protocols 2006, 1, 695-700). Cells expressing the fusion proteins are incubated with the appropriate CPP conjugate. TRM is used to quantitatively determine luminescence intensity in the cytosol and the plasma membrane or in stress fibers, and compared against unlabeled control cells. Quantitative image analysis is used to determine the signal of specifically and non-specifically bound probe above background. By observing cells over a period of hours, the in cellulo stability of the DHFR-TMP-LC complex is determined.

### Example 6

### In vivo TRM imaging of Lumi4®-Tb -to-GFP FRET

Here, eDHFR labeled with a TMP-terbium complex is used as a FRET donor and GFP as a FRET acceptor to dynamically visualize intramolecular and intermolecular FRET in living cells, including neurons. A fusion of eDHFR linked to GFP is expressed in fibroblast cells, and then the best methods of cytosolic delivery is used to deliver TMP-terbium complex to the cells. With this model, TRM parameters are optimized to detect FRET from eDHFR-bound TMP-terbium complex to GFP as long-lifetime (>50 μs) sensitized GFP emission (ca. 520 nm). As a demonstration of intermolecular FRET in non-neuron cells and neurons, the method of Hayashi and co-workers (Okamoto et al., Nature Protocols 2006, 1, 911-919; Okamoto et al., Nat. Neurosci. 2004, 7, 1104-1112) is adapted to visualize F-actin and G-actin equilibrium as FRET between eDHFR-β-actin and GFP-β-actin fusions.

NIH3T3 fibroblast cells are transfected with DNA encoding an eDHFR-GFP fusion protein. Expressing cells are loaded with TMP-terbium complex via CPP-mediated delivery and/or osmotic lysis/pinocytosis. TRM is used to image intramolecular FRET between eDHFR-bound TMP-terbium complex as sensitized GFP emission detected at 520 nm with a narrow-pass emission filter after a delay time ≧50 μs. By adding an excess of unconjugated TMP to the cell culture, TMP-terbium complex is displaced from the fusion protein and the overall magnitude of the FRET signal is measured (i.e., the FRET dynamic range).

This system is also used to establish software protocols to measure the sensitized GFP acceptor emission lifetime (520 nm) or terbium donor emission lifetime (620 nm). Analysis of donor and sensitized acceptor emission lifetimes is used to determine the relative concentrations of interacting and non-interacting protein species (Heyduk et al., Anal Biochem 2001, 289, 60-7). The amplitude of the donor lifetime decay profile is constant and reflects the concentration of donor present in the cell. The amplitude of the sensitized acceptor lifetime reflects the concentration of donor species interacting with the acceptor. The absolute concentration of the acceptor can be determined by direct excitation and suitable calibration of the microscope. The important thing to note is that all of these measurements can be made by analyzing the images of a single cell, allowing for a sensitive method for determining the stoichiometry of protein-protein interactions in vivo. Established methods are used to determine emission lifetimes by capturing a series of images at different delay intervals, and using the intensities at different areas of the decay curve to calculate the lifetimes at each pixel of the image (Elangovan et al., J. Microscopy 2002, 205, 3-14).

After establishing labeling and microscopy protocols for quantitatively imaging FRET between eDHFR-bound TMP-terbium complex and GFP, intermolecular TR-FRET imaging is demonstrated in fibroblasts and neuron cells. Hayashi and co-workers demonstrated FRET imaging of F-actin and G-actin equilibrium in living cells by expressing β-actin fused to CFP and YFP (Okamoto et al., Nature Protocols 2006, 1, 911-919; Okamoto et al., Nat. Neurosci. 2004, 7, 1104-1112). A bicistronic expression vector is constructed that will express equivalent amounts of eDHFR-β-actin and GFP-β-actin upon transfection into fibroblasts or neurons. Expressing cells are labeled with TMP-terbium complex and TRM is used to image FRET between fusion proteins co-localized in stress fibers (such as F-actin). Addition of Latrunculin A and/or jaspoklinolide can be used to modulate the equilibrium of F-actin and G-actin. As controls for FRET, excess TMP can be added to the cell culture to displace TMP-terbium complex, resulting in a loss of FRET signal. Acceptor (GFP) photobleaching is also done to confirm the FRET signal. Fixation followed by Texas Red-phalloidin staining will be used to confirm that the luminescence is, in fact, observed within actin stress fibers. As with other protocols described here, the method is first optimized in NIH3T3 fibroblasts before being performed in rat hippocampal or cortex neurons. Successful completion of the proof-of-principle experiments will make available reagents and microscopy protocols for imaging FRET in living mammalian cells, including neurons. These methods are applicable to any FRET imaging methodology that currently employs fluorescent protein probes (Miyawaki, Neuron 2005, 48, 189-199), and should substantially enhance sensitivity, dynamic range and quantitative analysis of protein-mediated biochemical processes in living mammalian cells.

### Example 7

Selective DHFR inhibitors (antifolates) other than TMP that could be utilized for biotechnological applications in mammalian cells were identified. DHFR has been the focus of intense drug discovery efforts for several decades (Kompis et al., Chem. Rev. 2005, 105: 593-620), and in recent years there has been considerable focus put on developing selective inhibitors against DHFRs from pathogenic organisms such as Pneumocystis carinii, Plasmodium falciparum, and Staphylococcus aureus (Chan et al., Curr. Med. Chem. 2006, 13: 377-398). Hundreds of TMP analogues (substituted 5-benzyl pyrimidines) have been prepared in efforts to analyze binding modes and to develop improved therapies against resistant organisms (Forsch et al., Bioorg. Med. Chem. Lett. 2004, 14: 1811-1815; Kuyper et al., J. Med. Chem. 1985, 28: 303-311; Kuyper et al., J. Med. Chem. 1982, 25: 1120-1122; Rosowsky et al., J. Med. Chem. 1999, 42: 4853-4860; Rosowsky et al., J. Med. Chem. 2002, 45: 233-241; Rosowsky et al., J. Med. Chem. 2003, 46: 1726-1736; Sirichaiwat et al., J. Med. Chem. 2004, 47: 345-354; Tarnchompoo et al., J. Med. Chem. 2002, 45: 1244-1252). Efforts were focused on compounds 2 and 3 (Scheme 8) which exhibit approximately nanomolar inhibitory activities against P. falciparum DHFR (pfDHFR) and P. carinii DHFR (pcDHFR), while they exhibit minimal or no activity against mammalian DHFRs (Forsch et al., Bioorg. Med. Chem. Lett. 2004, 14: 1811-1815; Sirichaiwat et al., J. Med. Chem. 2004, 47: 345-354; Tarnchompoo et al., J. Med. Chem. 2002, 45: 1244-1252).

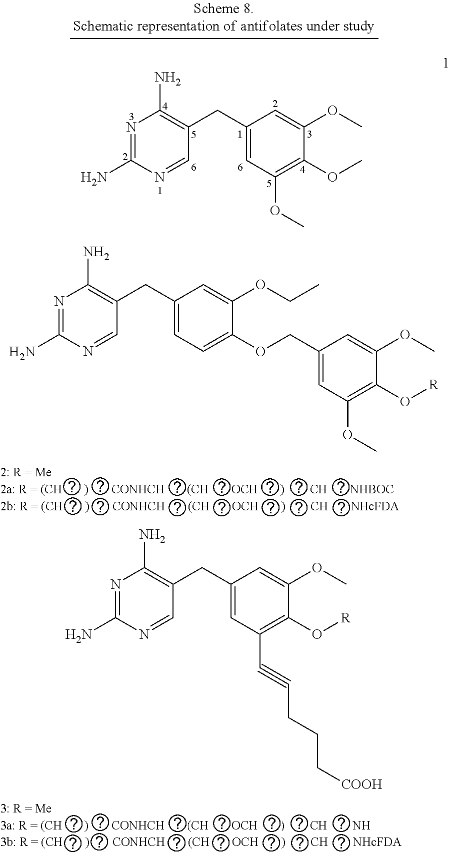

The common numbering scheme for 5-substituted benzyl pyrimidines is given for 1 (trimethoprim, TMP). Compounds 2b and 3b are heterodimeric conjugates of 2 and 3 to acetylated 5(6)-carboxy fluorescein (cFDA).

Syntheses of compounds 2a, 2b, 3a, 3b. The general scheme for preparation of the antifolate anlogs in this study first entailed aldol condensation of an appropriately substituted benzaldehyde with 3-morpholinopropanenitrile, followed by replacement of the morpholine leaving group with aniline and subsequent cyclization with guanidine to yield 5-benzyl pyrimidine scaffolds. (Schemes 9, 12). Further substitutions yielded analogs 2a, 3a, which could be conjugated to commercially available 5(6)-carboxyfluorescein diacetate to yield 2b, 3b (Schemes 11, 13).

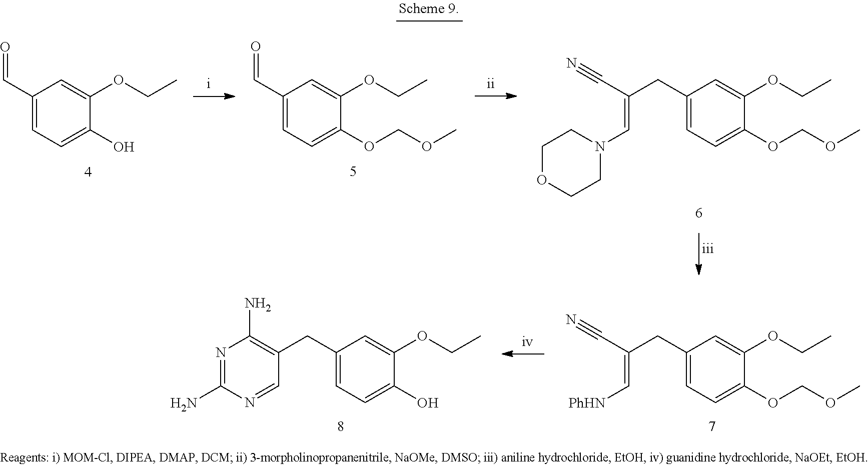

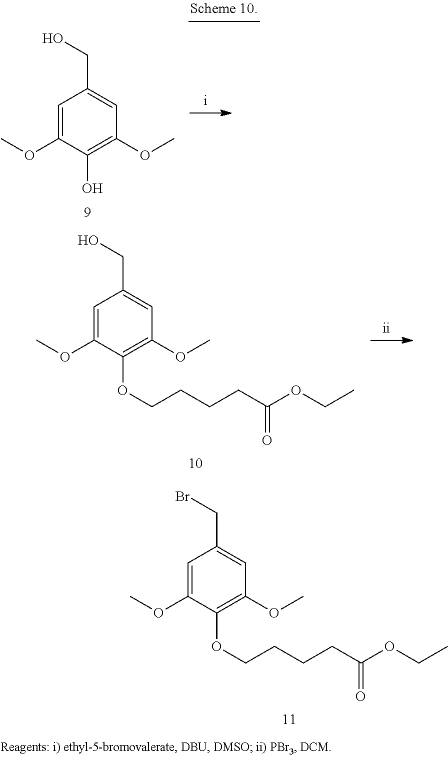

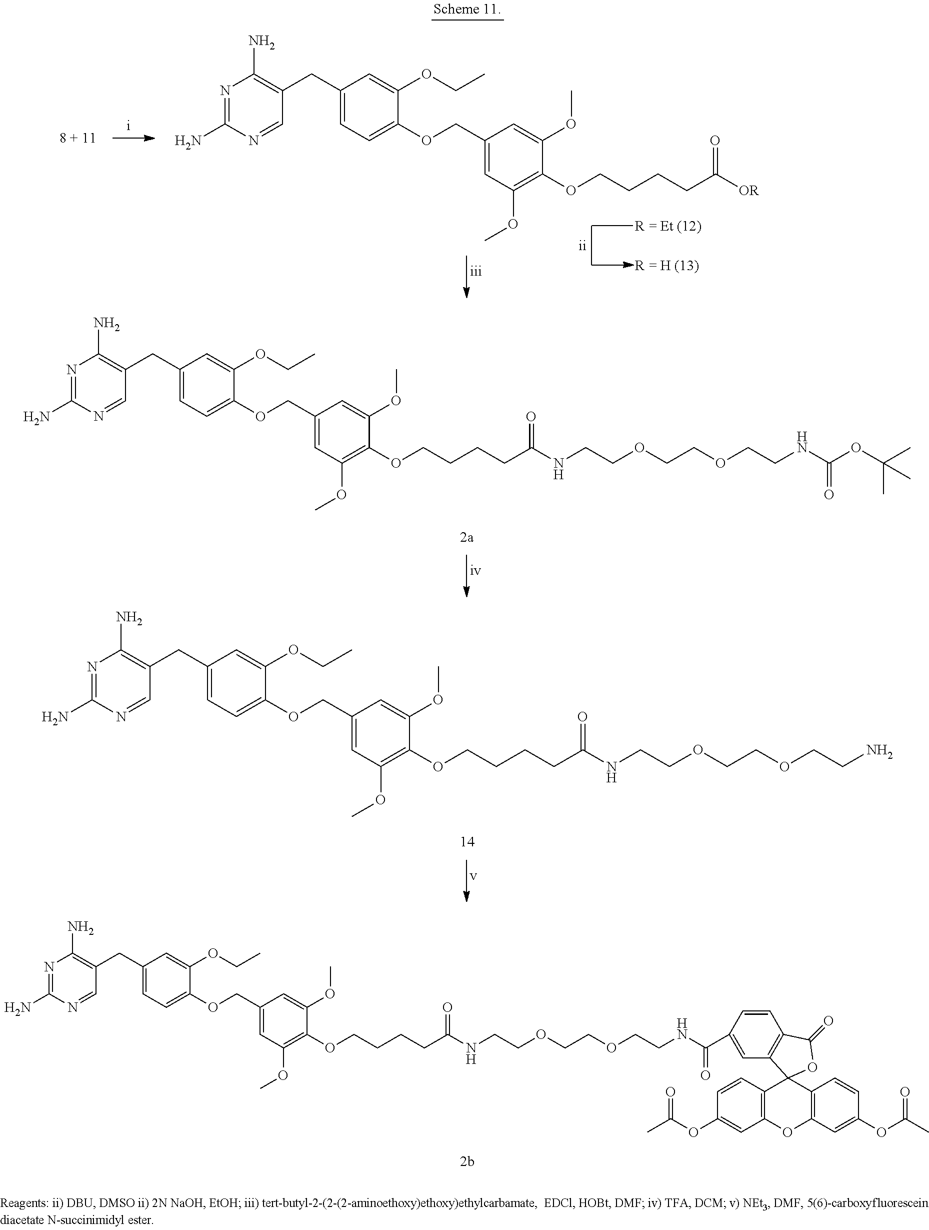

Synthesis of 2a, 2b: 3-Ethoxy-4-(methoxymethoxy)benzaldehyde (5): A magnetically stirred mixture of 3-ethoxy-4-hydroxybenzaldehyde (4, 4 gm, 24.1 mmol), DMAP (500 mg, 4.1 mmol), and DIPEA (3.73 gm, 28.91 mmol) in DCM (60 mL) maintained at 0° C. (ice-water bath) under an atmosphere of nitrogen was treated, dropwise, with MOM-Cl (2.31 gm, 28.9 mmol). The ensuing reaction mixture was allowed to warm to 18° C., stirred at this temperature for 18 hours and then poured into cold HCl (140 mL of a 0.1N aqueous solution). The separated aqueous layer was extracted with DCM (3×50 mL) and combined organic extracts washed with water (60 mL) and brine (60 mL) and then dried over MgSO₄, filtered, and concentrated under reduced pressure. The resulting solid was subjected to flash chromatography (3:7 ethyl acetate/hexane elution) to afford, after concentration of appropriate fractions 3-ethoxy-4-(methoxymethoxy) benzaldehyde (4.02 gm, 79%). ¹H NMR (400 MHz, CDCl₃) δ 1.46-1.60 (m, 3H), 3.52 (s, 3H), 4.15-4.18 (m, 2H), 5.31 (s, 2H), 7.25-7.27 (m, 1H), 7.39-7.42 (m, 2H), 9.85 (s, 1H); ¹³C NMR (125.7 MHz, CDCl₃) δ 14.6, 56.4, 64.4, 95, 110.8, 115.3, 126.1, 131.1, 149, 152.5; ESMS⁺ (m/z) 211 [M+H]⁺.

4-((2,4-Diaminopyrimidin-5-yl)methyl)-2-ethoxyphenol (8): Step 1. A solution of NaOMe was prepared by dissolving clean metallic Na (120 mg, 5.22 mmol) in anhydrous MeOH (10 mL). The solvent was evaporated under reduced pressure, and the solid was taken up in DMSO (15 mL), and to the solution was added 3-morpholinopropanenitrile (24) (2.20 gm, 15.71 mmol) at 65° C. The mixture was heated to 80° C. for 45 min, followed by the addition of 3-ethoxy-4-(methoxymethoxy)benzaldehyde (5), 3 gm, 14.28 mmol) in 15 mL of DMSO over a 45 minute period. After heating for 2.5 hours, the reaction mixture was cooled and partitioned between EtOAc and H₂O that had been slightly acidified with dilute aqueous citric acid to prevent the formation of an emulsion and extracted with EtOAc. The combined organic extracts washed with brine and then dried over MgSO₄, filtered, and concentrated under reduced pressure. The crude product was purified by flash chromatography (1:1 EtOAc/hexane elution) to afford the pure 2-(3-ethoxy-4-(methoxymethoxy)benzyl)-3-morpholinoacrylonitrile (6) (1.2 gm, 25%) as a yellow gum. ¹H NMR (400 MHz, CDCl₃) δ 1.41-1.45 (t, 3H, J=4 Hz), 3.30 (s, 2H), 3.40-3.47 (m, 4H), 3.51 (s, 3H), 3.60-3.72 (m, 4H), 4.06-4.13 (q, 2H, J=8, 16 Hz), 5.18 (s, 2H), 6.21 (s, 1H), 6.65-7.07 (m, 3H); ¹³C NMR (100.6 MHz, CDCl₃) δ 14.8, 38.9, 49.4, 56.1, 64.4, 66.2, 75.4, 95.8, 113.7, 117.6, 120.4, 121.7, 134, 145.4, 148.8, 149.4.

Step 2. A solution of 2-(3-ethoxy-4-(methoxymethoxy)benzyl)-3-morpholinoacrylonitrile (6, 1.2 gm, 3.61 mmol) and aniline hydrochloride (698 mg, 5.41 mmol) in anhydrous EtOH (15 mL) was refluxed for 1 hour. In a separate flask, guanidine hydrochloride (1.71 gm, 18 mmol) was added to a solution of NaOEt prepared by dissolving clean metallic Na (415 mg, 18.04 mmol) in anhydrous EtOH (20 mL), and the flask was swirled manually for 10 minutes. The entire contents of the second flask (including the NaCl) were added to the first, and the combined mixture was refluxed for 20 hours and then filtered while hot. Flash chromatography with 9:1 EtOAc/MeOH as the eluent afforded the pure product (500 mg, 53%). ¹H NMR (500 MHz, CD₃OD) δ 1.36-1.40 (t, 3H, J=5.0 Hz), 3.59 (s, 2H), 3.90-4.10 (m, 2H), 6.60-6.65 (m, 1H), 6.70-6.80 (m, 2H), 7.26 (s, 1H); ¹³C NMR (125.7 MHz, CD₃OD) δ 15.24, 32.64, 64.30, 106.86, 114.77, 115.83, 121.13, 131.10, 145.43, 146.88, 155.46, 162.39, 162.69; ESMS⁺ (m/z) 261 [M+H]⁺.

Ethyl 5-(4-(hydroxymethyl)-2,6-dimethoxyphenoxy)pentanoate (10): To a well stirred solution of 4-(hydroxymethyl)-2,6-dimethoxyphenol (9), 500 mg, 2.72 mmol) in DMSO (4 mL), was added DBU (496 mg, 3.26 mmol) dropwise at room temperature. After 30 minutes, ethyl-5-bromovalerate (681 mg, 3.26 mmol) was added slowly to the reaction mixture and stirred for 12 hours. Water (approximately 50 ml) was added and the product was extracted with EtOAc (3×50 mL). The combined EtOAc solution was washed with approximately 100 mL water, dried over MgSO₄, filtered and the solvent was evaporated under reduced pressure. The crude product was subjected to flash chromatography (3:7 EtOAc/hexane) to afford the pure product (704 mg, 83%). ¹H NMR (400 MHz, CDCl₃) δ 1.22-1.26 (m, 3H), 1.79-1.83 (m, 5H), 2.36-2.37 (m, 2H), 3.83 (s, 6H), 3.93-3.96 (m, 2H), 4.10-4.20 (m, 2H), 4.61-4.62 (m, 2H), 6.57 (s, 2H); ¹³C NMR (100.6 MHz, CDCl₃) δ 14.2, 21.4, 29.4, 33.9, 56, 60.1, 65.5, 72.6, 103.8, 136.4, 153.5, 174.

Ethyl-5-(4-(bromomethyl)-2,6-dimethoxyphenoxy)pentanoate (11): Phosphorous tribromide (835 mg, 3.09 mmol) was added to a solution of ethyl-5-(4-(hydroxymethyl)-2,6-dimethoxyphenoxy)pentanoate (10) (2.68 gm, 8.59 mmol) in dry DCM (60 mL) at 0° C. The mixture was stirred at room temperature for 1 hour before it was treated with cold water (30 mL). The layers were separated and the water phase extracted with DCM (3×50 mL). The combined organic layers were washed with water (50 mL), a saturated NaHCO₃ solution (50 mL), a saturated NaCl solution (50 mL), dried over MgSO₄, filtered, concentrated in vacuo and purified by column chromatography (2:8 EtOAc/hexane) to afford the pure bromide (11) (1.84 gm, 57%). ¹H NMR (500 MHz, CDCl₃) δ 1.23-1.26 (m, 3H), 1.78-1.90 (m, 5H), 2.35-2.42 (m, 2H), 3.84 (s, 6H), 3.92-3.98 (m, 2H), 4.10-4.20 (m, 2H), 4.45 (m, 2H), 6.60 (s, 2H); ¹³C NMR (125.7 MHz, CDCl₃) δ 14.2, 21.4, 29.4, 33.9, 34.3, 56.1, 60.1, 72.7, 106.2, 132.9, 137.4, 153.4, 173.6; ESMS+(m/z) 397 [M+Na]⁺.

Ethyl-5-(4-((4-((2,4-diaminopyrimidin-5-yl)methyl)-2-ethoxyphenoxy)methyl)-2,6-dimethoxyphenoxy) pentanoate (12): To a well stirred solution of 4-((2,4-diaminopyrimidin-5-yl)methyl)-2-ethoxyphenol (8) (460 mg, 1.77 mmol) in DMSO (5 mL), was added DBU (323 mg, 2.13 mmol) dropwise at room temperature. After 30 minutes, ethyl 5-(4-(bromomethyl)-2,6-dimethoxyphenoxy)pentanoate (11) (665 mg, 1.77 mmol) was added slowly to the reaction mixture and stirred for 12 hours. Water (approximately 50 mL) was added and the product was extracted with EtOAc (4×50 mL). The combined EtOAc solution was washed with approximately 100 mL water, dried over MgSO₄, filtered and the solvent was evaporated under reduced pressure. The crude product was subjected to flash chromatography (9:1 EtOAc/MeOH) to afford the pure product (390 mg, 40%). ¹H NMR (400 MHz, CD₃OD) δ 1.22-1.26 (m, 3H), 1.36-1.40 (m, 3H), 1.68-1.90 (m, 4H), 2.30-2.2.45 (m, 2H), 3.95-4.20 (m, 14H), 5.01 (s, 2H), 6.45-7.05 (m, 5H), 6.60 (s, 2H); 7.40 (s, 1H); ESMS⁺ (m/z) 555 [M+H]⁺.

5-(4-((4-((2,4-Diaminopyrimidin-5-yl)methyl)-2-ethoxyphenoxy)methyl)-2,6-dimethoxyphenoxy) pentanoic acid (13): To a stirred solution of ethyl-5-(4-((4-((2,4-diaminopyrimidin-5-yl)methyl)-2-ethoxyphenoxy)methyl)-2,6-dimethoxyphenoxy)pentanoate (12) (390 mg, 0.70 mmol) in EtOH (10 mL) 2 N NaOH aqueous solution (1.5 mL) was added dropwise and the reaction mixture was stirred at room temperature for 10 hours. Then the EtOH was removed under reduced pressure and the crude reaction mixture was adjusted to pH 4 with 1N HCl and extracted with EtOAc (6×50 mL). The combined organic extracts washed with brine and then dried over MgSO₄, filtered, and concentrated under reduced pressure (100 mg, 27%). The acid was used directly for the next step.

Synthesis of compound 2a: To a well stirred solution of 5-(4-((4-((2,4-diaminopyrimidin-5-yl)methyl)-2-ethoxyphenoxy)methyl)-2,6-dimethoxyphenoxy)pentanoic acid (13) (100 mg, 0.19 mmol) in DMF (3 mL) was added EDCI (43.55 mg, 0.23 mmol), HOBt (30.78 mg, 0.23 mmol) and tert-Butyl-2-(2-(2-aminoethoxy)ethoxy)ethylcarbamate (25) (56.58 mg, 0.23 mmol) and the mixture was stirred for 24 h at room temperature. The DMF was evaporated in vacuum, the residue was dissolved in ethyl acetate and washed with saturated sodium bicarbonate solution and then with brine solution. The combined organic phases were dried over MgSO₄; the solvent was removed under reduced pressure and purified by column chromatography (9:1 EtOAc/MeOH) to afford the pure compound (50 mg, 35%). ¹H NMR (400 MHz, CD₃OD) δ 1.30-1.41 (m, 12H), 1.65-1.80 (m, 4H), 2.20-2.35 (m, 2H), 3.18-3.22 (m, 2H), 3.25-3.65 (m, 8H), 3.75 (s, 6H), 3.85 (s, 2H), 3.90-4.08 (m, 4H), 5.02 (s, 2H), 6.55-6.95 (m, 5H), 7.38 (1H); ¹³C NMR (125.7 MHz, CD₃OD) δ 13.9, 22.2, 27.3, 29, 32.2, 35.2, 38.8, 39.8, 55.2, 64.4, 69.2, 69.6, 69.8, 71.1, 72.5, 104.5, 107.6, 114.3, 115.3, 120.6, 131.7, 133.3, 147.1, 149.3, 150.2, 153.3, 157.5, 160, 163.4, 174.8; ESMS⁺ (m/z) 757 [M+H]⁺.

Synthesis of compound 14: Trifluoroacetic acid (0.1 mL) was added to a solution of compound 2a (15 mg, 0.02 mmol) in dichloromethane (4 mL) at 0° C. The reaction mixture was stirred for 12 hours at room temperature. After the reaction was complete, the solvent was removed under reduced pressure at room temperature and used directly in the next step.

Synthesis of compound 2b: To a well stirred suspension of amine 14 (12 mg, 0.02 mmol) in DMF (2 mL), two drops of triethylamine was added. After 30 minutes, 5-carboxyfluorescein diacetate N-succinimidyl ester (12 mg, 0.02 mmol) was added and stirred the reaction mixture for 3 hours at room temperature. The solvent was removed under reduced pressure and extracted with ethyl acetate (4×30 mL). The combined organic phases were dried over MgSO₄, filtered; the solvent was removed under reduced pressure and purified by column chromatography (9:1 EtOAc/MeOH) to afford the pure compound (8 mg, 40%). ESMS⁺ (m/z) 1117 [M+H₂O+H]⁺.

3-Morpholinopropanenitrile (24): A mixture of morpholine (1 gm, 11.49 mmol) and acrylonitrile (609 mg, 11.49 mmol) was stirred at room temperature as a neat mixture without any solvent and catalyst for 3 hours. The reaction mixture was then straightway subjected to short column chromatography over silica gel (3:7 EtOAc/hexane) to provide the pure 3-morpholinopropanenitrile (1.2 gm, 75%). ¹H NMR (400 MHz, CDCl₃) δ 2.47-2.52 (m, 6H), 2.64-2.68 (m, 2H), 3.69-3.71 (m, 4H); ¹³C NMR (100.6 MHz, CDCl₃) δ 15.7, 53, 53.6, 66.7, 118.6.

tert-Butyl-2-(2-(2-aminoethoxy)ethoxy)ethylcarbamate (25): 2-(2-(2-Aminoethoxy) ethoxy) ethanamine (6 gm, 40.54 mmol) was dissolved in a solution of triethyl amine methanol (10% TEA in MeOH, 130 mL). A solution of di-tert-butyl dicarbonate (2.95 gm, 13.53 mmol) in methanol (10 mL) was added to this mixture with vigorous stirring. The mixture was refluxed for 2 hours and left to stir at room temperature overnight. The excess methanol and TEA were removed in vacuo to yield an oily residue that was dissolved in dichloromethane and washed with 10% aqueous sodium carbonate. The organic layer was separated, dried over MgSO₄ and filtered, and the solvent was removed in vacuo. The oily residue was filtered through a bed of silica and used directly in the next step.

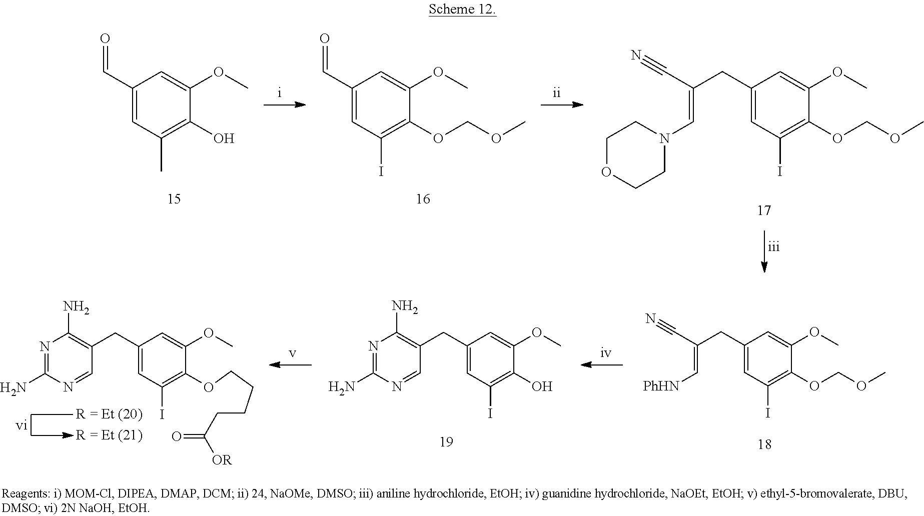

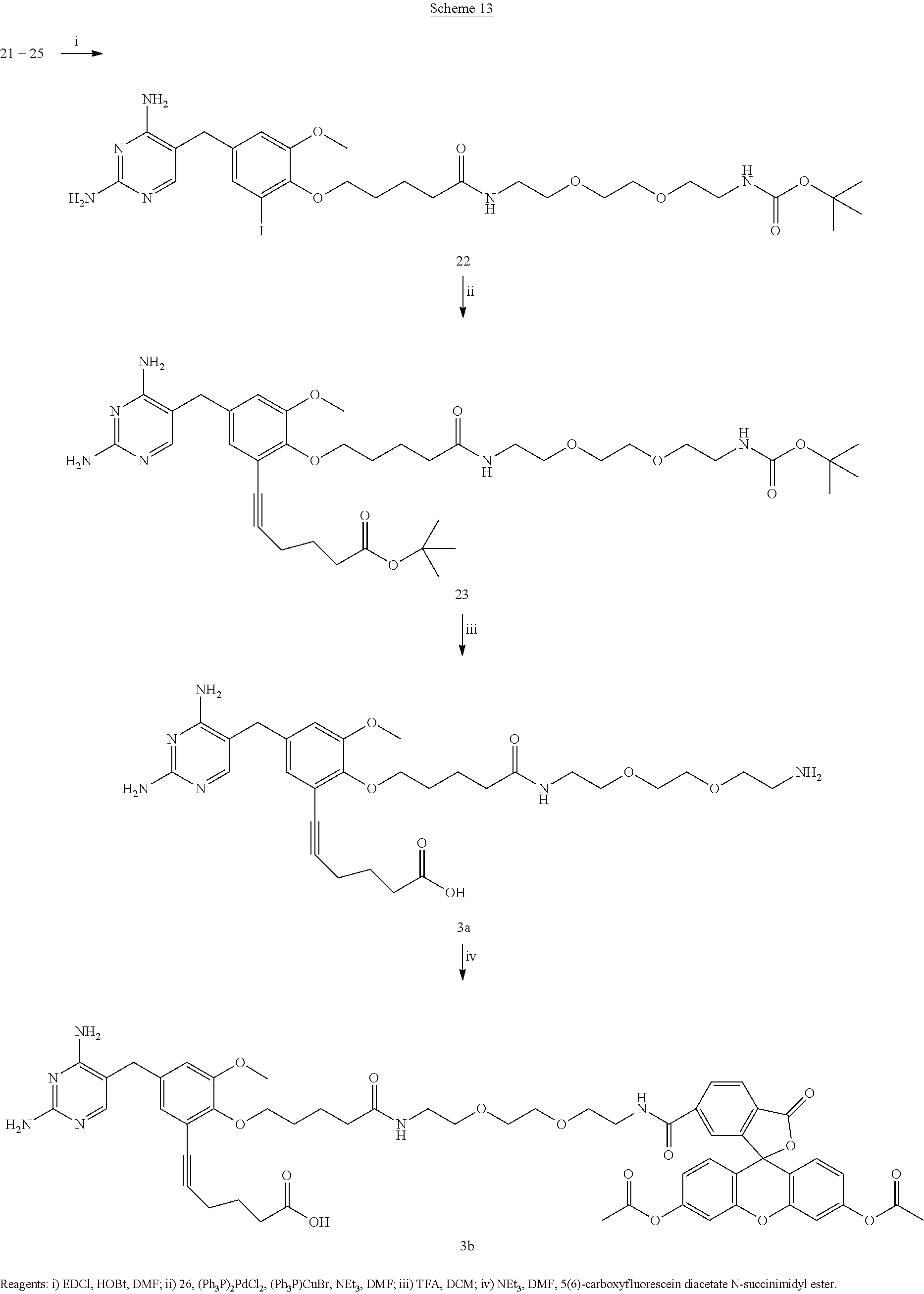

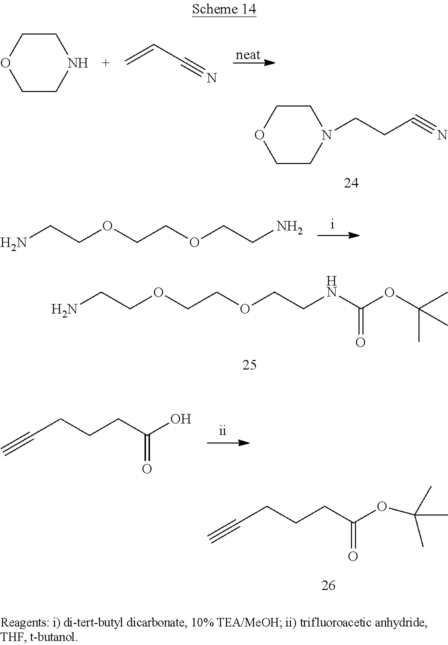

3-Iodo-5-methoxy-4-(methoxymethoxy)benzaldehyde (16): A magnetically stirred mixture of 5-iodovanillin(15) (3 gm, 10.79 mmol), DMAP (658 mg, 5.39 mmol), and DIPEA (1.67 gm, 12.95 mmol) in DCM (60 mL) maintained at 0 oC (ice-water bath) under an atmosphere of nitrogen was treated, dropwise, with MOM-Cl (1.04 gm, 13 mmol). The ensuing reaction mixture was allowed to warm to 18° C., stirred at this temperature for 18 hours and then poured into cold HCl (120 mL of a 0.1 N aqueous solution). The separated aqueous layer was extracted with DCM (3×50 mL) and combined organic extracts washed with water (60 mL) and brine (60 mL) and then dried over MgSO₄, filtered, and concentrated under reduced pressure. The resulting solid was subjected to flash chromatography (3:7 ethyl acetate/hexane elution) to afford, after concentration of appropriate fractions 3-iodo-5-methoxy-4-(methoxymethoxy) benzaldehyde (2.80 gm, 81%). ¹H NMR (500 MHz, CDCl₃) δ 3.66 (s, 3H), 3.97 (s, 3H), 6.68 (S, 2H), 7.37 (s, 1H), 7.81 (s, 1H), 9.77 (s, 1H); ¹³C NMR (125.7 MHz, CDCl₃) δ 56.5, 80.4, 98, 108.6, 131, 136, 146.4, 151.4, 189.4.

4-((2,4-Diaminopyrimidin-5-yl)methyl)-2-iodo-6-methoxyphenol (19): Step 1. A solution of NaOMe was prepared by dissolving clean metallic Na (120 mg, 5.22 mmol) in anhydrous MeOH (10 mL). The solvent was evaporated under reduced pressure, and the solid was taken up in DMSO (15 mL), and to the solution was added 3-morpholinopropanenitrile (24) (1.43 gm, 10.21 mmol) at 65° C. The mixture was heated to 80° oC for 45 min, followed by the addition of 3-iodo-5-methoxy-4-(methoxymethoxy)-benzaldehyde (16) (3 gm, 9.32 mmol) in 15 mL of DMSO over a 45 minute period. After heating for 2.5 hours, the reaction mixture was cooled and partitioned between EtOAc and H₂O that had been slightly acidified with dilute aqueous citric acid to prevent the formation of an emulsion and extracted with EtOAc. The combined organic extracts washed with brine and then dried over MgSO₄, filtered, and concentrated under reduced pressure. The crude product was purified by flash chromatography (1:1 EtOAc/hexane elution) to afford the pure 2-(3-iodo-5-methoxy-4-(methoxymethoxy)-benzyl)-3-morpholinoacrylonitrile (17) (900 mg, 22%) as a yellow gum. ¹H NMR (500 MHz, CDCl₃) δ 3.24 (s, 2H), 3.46-3.48 (m, 4H), 3.65 (s, 3H), 3.68-3.71 (m, 4H), 3.82 (s, 3H), 5.12 (s, 2H), 6.23 (s, 1H), 6.76 (s, 1H), 7.18 (s, 1H); ¹³C NMR (125.7 MHz, CDCl₃) δ 38.5, 49.4, 56, 58.3, 66.2, 74.2, 92.6, 98.6, 113.1, 121.4, 130.2, 137.6, 145, 149, 152.1; ESMS⁺ (m/z) 445 [M+H]⁺, 462 [M+NH3+H]⁺, 467 [M+Na]⁺.

Step 2. A solution of 2-(3-iodo-5-methoxy-4-(methoxymethoxy)benzyl)-3-morpholinoacrylonitrile (17) (900 mg, 2.03 mmol) and aniline hydrochloride (393 mg, 3.05 mmol) in anhydrous EtOH (15 mL) was refluxed for 1 hour. In a separate flask, guanidine hydrochloride (964 mg, 10.15 mmol) was added to a solution of NaOEt prepared by dissolving clean metallic Na (233 mg, 10.13 mmol) in anhydrous EtOH (15 mL), and the flask was swirled manually for 10 minutes. The entire contents of the second flask (including the NaCl) were added to the first, and the combined mixture was refluxed for 20 hours and then filtered while hot. Flash chromatography with 9:1 EtOAc/MeOH as the eluent afforded the pure product (400 mg, 53%). ¹H NMR (400 MHz, CDCl₃) δ 3.58 (s, 2H), 3.82 (s, 3H), 6.80 (s, 1H), 7.10 (s, 1H), 7.28 (s, 1H); ¹³C NMR (125.7 MHz, CDCl₃) δ 32, 56.4, 84.8, 106.2, 113, 129.6, 133.6, 144.7, 147.3, 156.1, 162.5, 162.6; ESMS⁺ (m/z) 373 [M+H]⁺.

Ethyl-5-(4-[2,4-d]aminopyrimidin-5-yl)methyl)-2-iodo-6-methoxyphenoxy)pentanoate (20): To a well stirred solution of 4-((2,4-diaminopyrimidin-5-yl)methyl)-2-iodo-6-methoxyphenol (19) in DMSO (5 mL), was added DBU (196 mg, 1.29 mmol) dropwise at room temperature. After 30 minutes, ethyl-5-bromovalerate (269 mg, 1.29 mmol) was added slowly to the reaction mixture and stirred for 12 hours. Water (approximately 50 ml) was added and the product was extracted with EtOAc (4×50 mL). The combined EtOAc solution was washed with approximately 100 mL water, dried over MgSO₄, filtered and the solvent was evaporated under reduced pressure. The crude product was subjected to flash chromatography (9:1 EtOAc/MeOH) to afford the pure product (450 mg, 83%). ¹H NMR (500 MHz, CDCl₃) δ 1.23-1.26 (t, J=5 Hz, 3H), 1.80-1.87 (m, 4H), 2.40-2.43 (t, J=10 Hz, 4H), 3.61 (s, 2H), 3.79 (s, 3H), 3.91-3.93 (t, J=5 Hz, 2H), 4.09-4.13 (q, J=5, 10 Hz, 2H), 6.86 (s, 1H), 7.16 (s, 1H), 7.50 (s, 1H); ¹³C NMR (125.7 MHz, CDCl₃) δ 13.1, 21.4, 29.1, 31.8, 33.5, 54.9, 59.9, 71.9, 91.7, 106.1, 113, 129.7, 137.1, 146.5, 152.5, 154.3, 161.7, 163, 174; ESMS⁺ (m/z) 501 [M+H]⁺.

5-(4-[2,4-d]aminopyrimidin-5-yl)methyl)-2-iodo-6-methoxyphenoxy)pentanoic acid (21): To a stirred solution of ethyl-5-(4-((2,4-diaminopyrimidin-5-yl)methyl)-2-iodo-6-methoxyphenoxy)pentanoate (20) (500 mg, 1 mmol) in EtOH (10 mL) 2 N NaOH aqueous solution (2 mL) was added dropwise and the reaction mixture was stirred at room temperature for 10 hours. Then the EtOH was removed under reduced pressure and the crude reaction mixture was adjusted to pH 4 with 1 N HCl and extracted with EtOAc (6×50 mL). The combined organic extracts washed with brine and then dried over MgSO₄, filtered, and concentrated under reduced pressure (300 mg, 64%). The acid was used directly for the next step.

Synthesis of compound 22: To a well-stirred solution of 5-(4-((2,4-diaminopyrimidin-5-yl)methyl)-2-iodo-6-methoxyphenoxy)pentanoic acid (21) (360 mg, 0.76 mmol) in DMF (6 mL) were added EDCI (174 mg, 0.91 mmol), HOBt (123 mg, 0.91 mmol) and compound 25 (189 mg, 0.76 mmol) and the mixture was stirred for 24 h at room temperature. The DMF was evaporated in vacuum, the residue was dissolved in ethyl acetate and washed with saturated sodium bicarbonate solution and then with brine solution. The combined organic phases were dried over MgSO₄, the solvent was removed under reduced pressure and purified by column chromatography (9:1 EtOAc/MeOH) to afford the pure compound (300 mg, 56%). ¹³C NMR (125.7 MHz, CDCl₃) δ 22.4, 27.4, 29.3, 31.6, 35.3, 38.9, 39.8, 55.1, 69.2, 69.6, 69.8, 72.2, 78.7, 92, 107.2, 113.2, 129.8, 136.1, 146.8, 148.2, 152.6, 163.6, 174.7; ESMS⁺ (m/z) 703 [M+H]⁺, ESMS⁺ (m/z) 725 [M+Na]⁺.

Synthesis of compound 23: A stirred mixture of 26 (14.31 mg, 0.08 mmol), iodide (22) (50 mg, 0.07 mmol), (Ph3P)2PdC12 (10 mg), (Ph₃P)₃CuBr (1 mg), and NEt₃ (1 mL) in dry DMF (4 mL) was heated at 60° C. for 72 hours. The solvent was removed in vacuum and extracted with ethyl acetate (4×50 mL). The combined organic phases were dried over MgSO₄, the solvent was removed under reduced pressure and purified by column chromatography (1:9 EtOAc/MeOH) to afford the pure compound (20 mg, 38%). ESMS⁺ (m/z) 743 [M+H]⁺, ESMS⁺ (m/z) 765 [M+Na]⁺.

Synthesis of compound 3a: Trifluoroacetic acid (4 mL) was added to a solution of the tert-butyl ester 23 (20 mg, 0.03 mmol) in dichloromethane (1 mL) at 0° C. The reaction mixture was stirred for 12 hours at room temperature. After the reaction was complete, the solvent was removed under reduced pressure at room temperature and used directly in the next step. ESMS⁺ (m/z) 587 [M+H]⁺, ESMS⁺ (m/z) 609 [M+Na]⁺.

Synthesis of compound 3b: To a well stirred suspension of 3a (20 mg, 0.03 mmol) in DMF (2 mL), three drops of triethylamine was added. After 30 min, 5-carboxy-fluorescein diacetate N-succinimidyl ester (20.05 mg, 0.04 mmol) was added and stirred the reaction mixture for 8 hours at room temperature. The solvent was removed under reduced pressure and filtered, washed with diethyl ether, dichloromethane and cold ethyl acetate. The solid was collected and dried in vacuum, and purified by preparative TLC (1:9 MeOH/EtOAc). ESMS⁺ (m/z) 1029 [M+H]⁺, ESMS⁺ (m/z) 1047 [M+H₃O]⁺.

Hex-5-ynoic acid tert-butyl ester (26): A dry flask was charged with hexynoic acid (1 gm, 8.93 mmol) and purged with nitrogen. THF (40 mL) was added, and the solution was cooled to 0° C. Trifluoroacetic anhydride (2.72 mL, 19.60 mmol) was added drop wise. The reaction was stirred at 0° C. for 2.5 hours, then t-butanol (3 mL) was added slowly. After 1 hour, the reaction was warmed to room temperature. The reaction was stirred for an additional 17 hours, quenched with water (50 mL) and extracted with ether (4×50 mL). The combined organic layers were dried with MgSO₄, filtered and concentrated. The resulting oil was purified by flash chromatography (3% EtOAc in hexane) yielding 14 (1.2 gm, 80%) as a clear oil. ¹H NMR (400 MHz, CDCl₃) δ 1.44 (s, 9H), 1.80 (pentet, J=7.2 Hz, 2H), 1.96 (t, J=2.7 Hz, 1H), 2.24 (dt, J=2.7, 7.0 Hz, 2H), 2.35 (t, J=7.5 Hz, 2H); ¹³C NMR (125.6 MHz, CDCl₃) δ 18.0, 24.0, 28.3, 34.4, 69.1, 80.5, 83.7, 172.7.

Conjugates of 2 and 3 (Scheme 8) to a hydrophobic, acetylated fluorescein were prepared (Scheme 8, (2b, 3b)). The conjugation strategy was informed by previously reported structural analyses that revealed the binding modes of each compound to their respective targets. Yuthavong and co-workers used semiempirical methods to model the binding of a close analogue of 2 to the known structure of pfDHFR (Sirichaiwat et al., J. Med. Chem. 2004, 47: 345-354). The 4′-trimethoxy-benzyl substituent was modeled to form a p-p stacking interaction with Phe116 of the wild-type pfDHFR. The para-methoxy is in van der Waals contact with Cys50 near the entrance to the binding pocket, and it was contemplated that alkyl linkage at this position would be the least disruptive to binding. The structure of pcDHFR in complex with 3 and NADPH was reported by Cody and co-workers (Cody et al., Proteins Struct. Funct. Bioinf. 2006, 65: 959-969). The 5′-(5-carboxy-1-pentynyl side chain interacts with Arg75, and this contributes strongly to the high affinity. The 4′-methoxy group interacts with the hydrophobic side chains of Leu25 and Ile33 near the entrance to the binding pocket, and this position was chosen for conjugation.

The conjugation strategy was validated by directly characterizing the ability of the nonfluorescent analogues 2a and 3a to inhibit Toxoplasma gondii DHFR (tgDHFR), a protozoan DHFR used as a model for biochemical characterization of pfDHFR inhibitors (Reynolds et al., J. Biol. Chem. 1998, 273: 3461-3469) and pcDHFR. By using an assay based on measurement of the change in absorbance at 340 nm when dihydrofolate is reduced to tetrahydrofolate in the presence of NADPH, inhibition constants for each analogue were determined (Rosowsky et al., J. Med. Chem. 1999, 42: 4853-4860). Methotrexate (MTX) and TMP were included in the assay as controls. 2a potently inhibited tgDHFR (IC₅₀=0.032 mm), while it poorly inhibited rat liver DHFR (IC₅₀=6 mm). 3a also exhibited excellent potency and selectivity, and strongly inhibited pcDHFR (IC₅₀=0.025 mm), while it showed about 350-fold lower activity against mammalian DHFR. The inhibition data suggested that heterodimers of 2 and 3 linked at the indicated positions would retain effective potency against their putative targets, while maintaining sufficient selectivity for applications in mammalian cell lines.

E. coli growth inihibition assay. E. coli (strain DH5α) was streaked onto Luria broth/agar plates containing varying concentrations of compounds 1 (TMP) 2a or 3a, and the plates were incubated at 37° C. for 24 hours. The minimal inhibitory concentration (MIC) was reported as the lowest concentration at which no colonies formed.

The linker-substituted analogues, 2a and 3a were further characterized by determining their ability to inhibit E. coli growth. The minimum inhibitory concentrations (MIC) of TMP, 2a and 3a were about 0.2 μm, 20 μm and >100 μm, respectively. The affinity of 2a for eDHFR relative to that of TMP was then determined. Compound 2 is about 30-fold and about 100-fold more potent than TMP against wild-type pfDHFR and the TMP-resistant double mutant (C59R, S108N), respectively. Because an analogue of 2 is substantially less potent against eDHFR than TMP, it is possible that 2b or other heterodimers of 2 could be used simultaneously with TMP for assays in mammalian cells. In the case of 3a, we expected that it would strongly inhibit eDHFR, as a 5′-(5-carboxy-alkyloxy)-TMP analogue was shown to be about 50-fold more potent against eDHFR than TMP itself (Kuyper et al., J. Med. Chem. 1985, 28: 303-311). The fact that 3a does not inhibit E. coli growth up to the concentrations tested suggests the molecule cannot cross the cell membrane. This was not entirely surprising as the carboxyl group of 3a is likely deprotonated, and localized charges typically lower the membrane permeability of small molecules (Calloway et al., ChemBioChem 2007, 8: 767-774).

Epifluorescence microscopy was used to determine the cell permeability and subcellular distribution of 2b and its selective binding to a soluble, TMP-resistant mutant (K27E, C59R, S108N) of the DHFR domain of P. falciparum DHFR-thymidylate synthetase (Japrung et al., Protein Eng. Des. Sel. 2005, 18: 457-464). An expression vector that targeted pfDHFR to the nucleus was prepared.

Site directed mutagenesis (QuickChange™ Multi-sitedirected mutagenesis kit, manufacturers instructions) was used to introduce mutations into plasmid pfDHFR(K27E)-GFP(C172T, T319A, C320A, G321T) to yield DNA encoding pfDHFR (K27E, C59R, S108N)-GFP. The genes for pfDHFR (K27E C59R S108N, 693 bp) and pcDHFR (615 bp) were inserted between the AgeI and XbaI restriction sites of the vector pLL1-NLS (Active Motif, Inc., Carlsbad, Calif.), yielding expression vectors that constitutively express pfDHFR (K27E, C59R, S108N) and pcDHFR as C-terminal fusions to three copies of the simian virus 40 large T-antigen nuclear localization sequence (DPKKKRKV; SEQ ID NO: 7).

Targeting was achieved by encoding pfDHFR soluble domain with a N-terminal fusion of three copies of the canonical simian virus 40 large T-antigen nuclear localization sequence (DPKKKRKV (SEQ ID NO: 7); Miller et al., Nat. Methods 2005, 2:255-257). NIH 3T3 fibroblast cells were then transfected with the vector. Approximately 24 hours after transfection, the cells were incubated with low (500 nm) concentrations of 2b and imaged microscopically.

Fluorescent labeling of pfDHFR fusion constructs: NIH3T3 fibroblast cells were cultured in Dulbecco's modified Eagle medium (DMEM) supplemented with fetal bovine serum (FBS; 10%), L-glutamine (2 mM), penicillin (100 IU μL-1), streptomycin (100 mg mL-1), HEPES (15 mM), and incubated in a humidified atmosphere at 37° C. and 5% CO₂. Cells (ca. 80,000) were seeded into 6-well plates, and transient transfection was performed by using Lipofectamine-2000™ reagent according to the manufacturer's protocol (2 μg DNA 6 μL Lipofectamine-2000™). After ca. 6 hours, the transfected cells were trypsinized and aliquoted (ca. 14,000 cells/well) into 8-well chambered coverslips (Nunc, Lab-Tek) and allowed to incubate another 12-18 hours. For imaging, fluorescein conjugates (2b, or 3b) were diluted (500 nM) in culture medium and incubated with the cells for ca. 15 minutes at 37° C. The cells were then washed 2× with PBS, and indicator-free DMEM without small molecule was added to the cells

Diffuse fluorescence was observed in all cells incubated with 2b, and some of the cells exhibited distinct nuclear fluorescence with more brightly fluorescent nucleoli, characteristic of the nucleus-targeting sequence (Miller et al., Nat. Methods 2005, 2:255-257). The diffuse fluorescence indicates that 2b readily enters cells, where the fluorescein moiety is hydrolyzed by intracellular esterases; this yields the fluorescent fluorescein dianion. Nuclear staining was attributed to the specific binding of 2b to nucleus-targeted pfDHFR soluble domain. Analogous imaging experiments were performed with 3b and cells transfected with a vector encoding pcDHFR fused to the N-terminal nucleus localization sequence. However, no intracellular fluorescence or nuclear staining with 3b was observed; this provided further evidence that analogues of 3 cannot passively diffuse into cells due to the presence of the 5′-(5-carboxy-1-pentynyl) moiety.

Microscopy. Epi-fluorescent microscopy of adherent live cells was performed using a Zeiss Axiovert 200 equipped with a 63X EC Plan Neofluar oil immersion objective (NA=1.25). Excitation illumination was provided by a 100 W Hg lamp. Excitation and emission light were selected by appropriate band-pass filters (Chroma Technologies, Inc. HQ480/40 (ex.), HQ535/50 (em.)). Images were detected using a Zeiss Axiocam MRM CCD camera, and captured with Zeiss Axiovision 4.6 software. Images were adjusted for brightness and contrast using NIH Image J and prepared for publication using Adobe Photoshop 5.5.

Enzyme inhibition assay. Compounds 2a and 3a were screened for their activity against a panel of purified DHFRs using an absorption-based inhbition assays (Rosowsky et al., J. Med. Chem. 1999, 42: 4853-4860). The assay was based on measurement of the change in absorbance at 340 nm when dihydrofolate is reduced to tetrahydrofolate in the presence of NADPH.

Substituted analogues of the established antifolates 2 and 3 retain similar potency and selectivity of the parent compounds when assessed in an inhibition assay. Compound 2a, a heterodimeric conjugate of 2 to a hydrophobic, acetylated fluorescein passively diffused into mammalian cells and selectively labeled a recombinantly expressed fusion of the soluble domain of pfDHFR. These results demonstrate that the considerable efforts devoted to finding selective inhibitors of pathogenic DHFRs can be leveraged to identify and develop new tools for chemical biology.

### Example 7

### Intracellular Probe Delivery and Specific Protein Labeling

Like many exogenous probes, such as quantum dots(Jaiswal et al., Nat Biotechnol 21(1):47-51 (2003)) or nucleic acid hybridizing agents, (Santangelo et al., Nat Methods 6(5):347-349 (2009)) TMP-Lumi4 will not diffuse passively into cells from culture medium. Therefore, to perform intracellular TR-FRET imaging, it was first necessary to establish methods of cytoplasmic probe delivery. Microinjection is one possible approach for loading adherent cells, and it has been successfully used for LC delivery.(Hanaoka et al., J Am Chem Soc 129(44):13502-13509 (2007)). However, microinjection requires specialized apparatus and can only be used to load relatively few cells at a time. Two techniques were therefore adapted to simultaneously deliver TMP-Lumi4 to the cytoplasm of many cells: 1) reversible plasma membrane permeabilization with Streptolysin O (SLO) (Ahnert-Hilger et al., Journal of Neurochemistry 52(6):1751-1758 (1989)); and 2) osmotic lysis of pinocytic vesicles (Okada et al., Cell 29(1):33-41 (1982)). Both methods allowed >50% loading efficiency while maintaining approximately 95% cell viability 2 hours post-treatment.

Probe Delivery via Streptolysin O (SLO)-mediated Membrane Permeabilization. SLO (1 mg/mL in PBS/50% glycerol, MBL International, Inc.) was diluted to a final concentration of 1000 ng/mL in 10 mM DTT/PBS and incubated at 37° C. for 2 hours. The pre-activated SLO was aliquoted and stored at −20° C. for later use. In a typical experiment, terbium-chelated TMP-Lumi4 was diluted to 15 μM in 100 μL Hank's Buffered Salt Solution (HBSS). Pre-activated SLO was added to a final concentration of 50 ng/mL (1:20 dilution of pre-activated SLO solution). NIH3T3 or MDCK cells in a single well of an 8-well chambered slide were washed 3× with pre-warmed (37° C.) HBSS. Then, 150 μL of pre-warmed TMP-Lumi4/SLO/HBSS solution was added, and the cells were incubated at 37° C. and 5% CO₂ for exactly 10 minutes. After incubation, 300 μL of DMEM containing 1.8 mM Ca²⁺ was added to the cells to effect resealing of membranes. The cells were incubated for at least 1 hour at 37° C. and 5% CO₂ before washing 3× with PBS and immersion in DMEM prior to imaging.

Probe Delivery via Osmotic Lysis of Pinosomes. A 6 μL aliquot of TMP-Lumi4 (300 μM in H₂O) was combined with approximately 1.2 equivalents of TbCl₃ (in approximately 3 μL H₂O), vortexed for 5 minutes, and allowed to stand at room temperature for 30 minutes. This step effects chelation of terbium, rendering the probe luminescent. The metal-labeled TMP-Lumi4 solution (approximately 9 μL) was combined with 27 μL of hypertonic growth medium (Influx™ reagent, Invitrogen, prepared according to manufacturer's instruction). NIH3T3 or MDCK cells in a single well of an 8-well chambered slide were washed 1× with pre-warmed (37° C.) PBS and 2× with pre-warmed hypertonic solution, respectively. Then, pre-warmed hypertonic solution containing TMP-Lumi4 was added, and the cells were incubated at 37° C. and 5% CO₂ for exactly 10 min. The cells were then quickly washed 2× with hypotonic solution (Influx™ reagent, Invitrogen, prepared according to manufacturer's instruction) and allowed to incubate in hypotonic solution for exactly 2 min. at room temperature to effect lysis of pinosomes. The cells were then washed 2× with PBS, immersed in complete DMEM and incubated for ˜1 h at 37° C. and 5% CO₂ before imaging.

Cell Viability Assay. A standard assay for cell viability (Live-dead™ assay, Invitrogen, Inc., L3224) was used to assess the effects of SLO and osmotic lysis of pinosomes on MDCK and NIH3T3 cells. Three separate experiments were performed for each cell type/treatment protocol, and the total number of living and dead cells was summed. Greater than 93% of cells were alive 2 hours after treatment (n>600 for each condition).

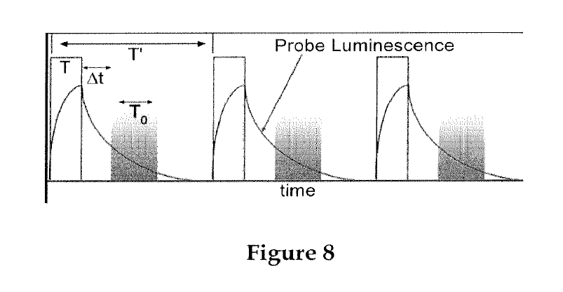

Intracellular delivery of TMP-Lumi4 and specific labeling of eDHFR fusion proteins was visualized using time-resolved microscopy. A conventional epi-fluorescence microscope was adapted for time-resolved imaging by incorporating a fast-modulated, UV LED (λₑₘ=365 nm) as the excitation source and an intensified CCD camera for image acquisition. The image intensifier component of the camera served as both a fast shutter and emission signal amplifier. By synchronizing the LED and intensifier with a digital delay generator, the excitation pulse width (T), delay time between pulse and detection (Δt), intensifier on-time (T₀) and pulse period (T′) could be varied independently (FIG. 8). Multiple excitation/detection cycles could be generated during a single camera frame with the camera control software summing multiple frames.

Live Cell Imaging. Microscopy of adherent live cells was performed using an epi-fluorescence microscope (Zeiss Axiovert 200M) modified with the following components: 1) a fast-modulated UV LED emitting at 365 nm (UV-LED-365, Prizmatix, Ltd.); 2) delay generator (DG645, Stanford Research Systems); 3) a gated image-intensified CCD camera (ICCD, mounted on the side-port of the microscope) and camera controller (Mega-10EX, Stanford Photonics, Inc.); and 4) a computer running Piper Control software (v2.4.05, Stanford Photonics, Inc.). A 100 W mercury arc lamp was available for continuous wave fluorescence excitation, and a conventional CCD (Zeiss Axiocam MRM) was mounted on the front port of the microscope. Filter cubes containing the appropriate excitation and emission filters and dichroics allowed for wavelength selection. Samples were imaged with a 63×/1.25 N.A. EC Plan Neofluar oil-immersion objective (Carl Zeiss, Inc.). For continuous-wave fluorescence and bright field images, the ICCD was set to “Live” mode, with automatic gain level and acquisition time.

For time-resolved microscopy with pulsed, near-UV excitation, image acquisition was initiated by a start signal (TTL) from the computer to the delay generator. Separate outputs (TTL) routed from the delay generator to the UV LED and the ICCD (via the camera controller) relayed a preprogrammed “burst” sequence to trigger the LED and the intensifier a user-defined number of times. For each acquisition, the signal from multiple excitation/emission events was accumulated on the ICCD sensor and read out to the image capture card of the computer at the end of the camera frame. The UV LED pulse width and pulse period, the intensifier delay time and on-time, the camera frame length (66.67 ms-2 s) and the intensifier gain voltage could be varied independently. The source/camera timing parameters were the same for all of the time-resolved images presented here: excitation pulse width=1500 μs, pulse period=3000 μs, delay time=100 μs, intensifier on-time=1390 μs. The camera control software allowed for summing multiple frames. Images (tagged image file format, .TIF) were captured with Piper control software and rendered using NIH Image J. Micrographs showing time-resolved fluorescence images and their associated controls were presented with identical contrast levels.

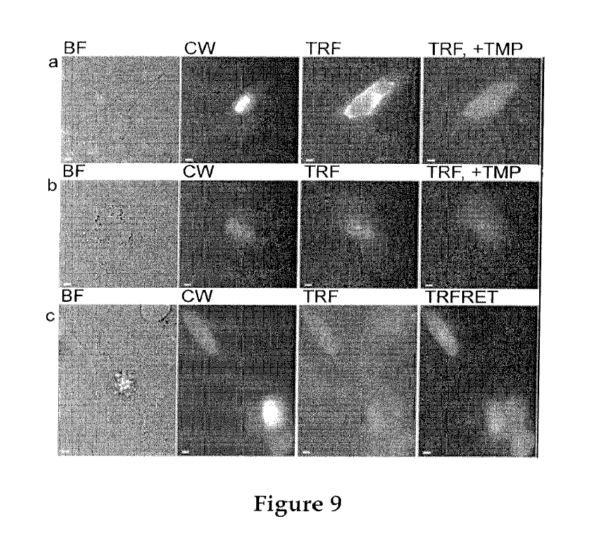

NIH3T3 fibroblast cells were transiently co-transfected with two plasmid DNA vectors; one that expressed plasma membrane targeted eDHFR and another that expressed nucleus-localized CFP as a positive control for transfection. After SLO-mediated delivery of TMP-Lumi4, timeresolved imaging revealed specific localization of terbium luminescence in the plasma membrane of a transfected cell loaded with probe (FIG. 9a). TMP (−10 μM) added to the imaging medium diffused into cells, competed with TMP-Lumi4 for eDHFR binding, and markedly diminished membrane luminescence (FIG. 9a). Similarly, Madin Darby canine kidney (MDCK) epithelial cells co-expressing nuclear-targeted CFP and nuclear-targeted eDHFR and loaded with TMP-Lumi4 using SLO displayed a specific nuclear, TR-FRET signal. Addition of TMP almost completely eliminated the TR-FRET signal from the nucleus (FIG. 9b).

Cell Culture. NIH 3T3 and MDCK cells were cultured in Dulbecco's Modified Eagle Media (DMEM, Invitrogen) supplemented with 10% FBS, 2 mM L-glutamine, 100 unit/ml penicillin and 100 mg/ml of streptomycin at 37° C. and 5% CO₂. NIH 3T3 and MDCK cells were passaged using 0.05% trypsin/0.03% EDTA solution (GIBCO) and 0.25% trypsin/0.03% EDTA solution, respectively.

Plasmids. Plasmids pLM1301 (expressing nucleus-localized CFP) and pLM1208 (expressing plasma membrane-localized eDHFR) were described previously (Miller et al., Nat Methods 2(4):255-257 (2005)). GFP-cldn1/tail was created by cloning amino acids 187-211 of human claudin-1 into pEGFP-C1 (Clontech). GFP-cldn1/tailΔYV was generated by point mutation to create a premature stop codon. ZO-1/PDZ1-eDHFR was created by inserting amino acids 19-113 of human ZO-1 (preceded by a start codon) into pLL-1NLS (Active Motif, Inc.) in frame with eDHFR. The integrity of all plasmids was verified by direct sequencing.

Cell Transfection. NIH3T3 or MDCK cells were seeded at 10⁵ cells per well into a 6-well plate. After approximately 18 hrs incubation at 37° C. and 5% CO₂, adherent cells (approximately 80% confluent) were transfected with 2 μg of the desired plasmid DNA using Lipofectamine-2000™ transfection reagent (Invitrogen) according to manufacturer's instructions. Approximately 6 hours after transfection, cells were trypsinized and reseeded at 14,000 cells/well into 8-well chambered slides and incubated at 37° C. and 5% CO₂ overnight.

To assess the sensitivity of terbium-to-GFP FRET in time-resolved mode, NIH3T3 fibroblasts were transfected with DNA encoding a GFP-eDHFR fusion protein. Adherent cells were loaded with TMP-Lumi4 by pinocytic delivery, as evidenced by the time-resolved image taken through a long-pass emission filter (>400 nm, FIG. 9c). Intramolecular, terbium-sensitized GFP emission is seen only in GFP-eDHFR-expres sing cells when visualized in time-resolved mode through a narrow-pass filter (λₑₘ=520±20 nm, FIG. 9c). The signal-to-noise ratio for intramolecular TR-FRET was calculated as the background subtracted mean of a cell image divided by the pixelwise standard deviation of a background region of equivalent area (Wolf et al., Methods Cell Biol 81:365-396 (2007)). The mean signal-to-noise ratio for a 9-cell sample (21.8±3.2, mean±s.d.) exceeded the FRET dynamic range for the brightest CFP/YFP FRET pairs in cell-free systems (approximately 5-fold) (Nguyen et al., Nat Biotechnol 23(3):355-360 (2005)). These results and those above demonstrate that both SLO-mediated membrane permeabilization and pinocytosis can effectively deliver TMP-Lumi4 into cells, and that the probe diffuses freely throughout the cytoplasm and nucleus without detectable non-specific binding. Furthermore, TMP-Lumi4 binds to eDHFR in cells, and time-resolved microscopy can visualize intramolecular terbium-to-GFP FRET as long-lifetime (>100 μs), sensitized GFP emission.

Signal-to-Noise Ratio Calculation. In order to assess the sensitivity of TR-FRET microscopy, the signal-to-noise ratio was calculated according to the equation:

signal-to-noise ratio=(μ_{signal}−μ_{bckg})/σ_{bckg},

where, μ_{signal} is equal to the mean pixel gray value in a region of interest (ROI) corresponding to the area of a cell, μ_{bckg} is equal to the mean pixel gray value in a nearby ROI of equivalent area, and μ_{bckg} is equal to the standard deviation of the pixel gray level in the background ROI (Wolf et al., Methods Cell Biol 81:365-396 (2007)). Because the signal level varies substantially within a single cell (e.g., between cytosol and nucleus), μ_{bckg} was chosen as representative of image noise. Signal-to-noise ratio analysis was performed on TR-FRET micrographs (λex=365 nm, λem=520±10 nm). Cells were selected that exhibited both GFP expression and loading of TMP-Lumi4 as determined by examining corresponding continuous wave fluorescence images and time-resolved fluorescence images of broad-band (λₑₓ=365 nm, λₑₘ>400 nm) emission. Signal-to-noise ratio was calculated for intermolecular TR-FRET in cells expressing interacting proteins (ZO-1/PDZ1-eDHFR and GFPcldn1/tail) and in cells expressing putatively non-interacting proteins (ZO-1/PDZ1-eDHFR and GFP-cldn1/tailΔYV). Signal-to-noise ratio was also calculated for intramolecular TR-FRET in cells expressing GFP-eDHFR. The mean, standard deviation and range of the signal-to-noise ratio was determined for each sample. P-value was determined from a two-tailed, two-sample, unequal variance t-test of the interacting and putatively non-interacting, intermolecular TR-FRET samples.

### Example 8

### TR-FRET Imaging of Protein-Protein Interactions

The ability of this FRET system to image protein-protein interactions was next used to measure the association of a PDZ domain with a carboxyl-terminal binding motif in living cells.(Songyang et al., Science 275(5296):73-77 (1997); Doyle et al., Cell 85(7):1067-1076 (1996)). Such PDZ-mediated interactions are fundamental to biological function in species from Drosophila to mammals. In epithelia, a direct interaction between the C-terminal cytoplasmic tail of claudin-1 and the most N-terminal PDZ domains (PDZ1) of ZO-1 and the related proteins ZO-2 and ZO-3 has been demonstrated using isolated recombinant proteins (Itoh et al., J. Cell. Biol. 147(6):1351-1363 (1999)). Moreover, PDZ1 domains of ZO-1, ZO-2, and ZO-3 are recruited to sites of claudin-1 polymerization in transfected fibroblasts (Itoh et al., J. Cell. Biol. 147(6):1351-1363 (1999)) but not to sites enriched in mutant claudin-1 lacking the C-terminal YV motif (Itoh et al., J. Cell. Biol. 147(6):1351-1363 (1999)). The relevance of this interaction to disease is emphasized by the observations that mutations within the ZO-2 PDZ1 domain are linked to familial hypercholanemia (Carlton et al., Nature Genetics 34(1):91-96 (2003)), a disease of hepatic bile transport, while loss of claudin-1 expression is associated with neonatal sclerosing cholangitis (Hadj-Rabia et al., Gastroenterology 127(5):1386-1390 (2004)), another hereditary cholestatic disease. However, a direct interaction between the C-terminal claudin-1 YV motif and the first PDZ domain of ZO-1 has not been previously demonstrated in live cells.

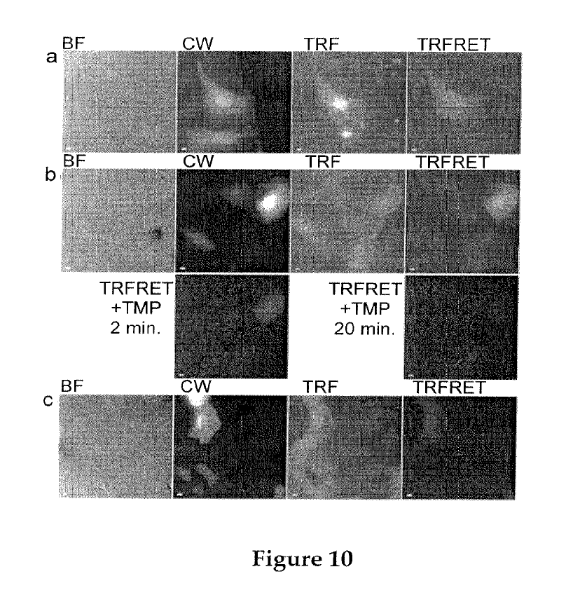

To examine the interaction between claudin-1 and ZO-1 in living cells, MDCK cells were transfected with expression vectors encoding a C-terminal fusion of eDHFR to the PDZ1 domain (residues 19-113) of ZO-1 (ZO-1/PDZ1-eDHFR) and an N-terminal fusion of EGFP to the C-terminal cytoplasmic domain (residues 187-211) of claudin-1 (GFP-cldn1/tail). Transfected cells were loaded with TMP-Lumi4 using both SLO-mediated and pinocytotic delivery methods. In both cases, TR-FRET imaging revealed terbium-sensitized GFP emission only in transfected cells loaded with TMP-Lumi4, providing an unambiguous image of protein-protein interaction (FIG. 10a,b). The signal-to-noise ratio for intermolecular TR-FRET was found to be 6.3±2.3 (mean±s.d.). Addition of excess TMP to growth medium eliminated the TR-FRET signal (FIG. 10b), further confirming that long-lifetime, sensitized GFP emission results from specific binding of TMP-Lumi4 to ZO-1/PDZ1-eDHFR. The predominantly nuclear localization of interactions between ZO-1/PDZ1-eDHFR and the GFP-cldn1/tail is consistent with previous reports showing that GFP-tagged ZO-1 PDZ1 domain accumulates within the nucleus (Itoh et al., J. Cell. Biol. 147(6):1351-1363 (1999)) and that ZO-1 directs subcellular claudin trafficking (Umeda et al., Cell 126(4):741-754 (2006)).

To assess specificity of the observed interaction, a GFP-cldn1/tail construct lacking the C-terminal YV motif (GFP-cldn1/tail^{ΔYV}) was developed and expressed in MDCK cells with ZO-1/PDZ1-eDHFR. While continuous widefield GFP fluorescence and broadband, time-resolved fluorescence signals were easily detected in cells loaded with TMP-Lumi4, only extremely faint TR-FRET was seen (FIG. 10c), verifying that the TR-FRET signal observed between ZO-1/PDZ1-eDHFR and the GFP-cldn1/tail reflects a specific PDZ-mediated interaction. There was a highly significant (P=0.001), >4-fold difference between the TR-FRET signal from ZO-1/PDZ1-eDHFR and GFP-cldn1/tail (signal-to-noise ratio=6.3±2.3) and that seen from ZO-1/PDZ1-eDHFR and GFP-cldn1/tail^{ΔYV} (signal-to-noise ratio=1.5±0.8).

The data above show that TR-FRET imaging is faster, more sensitive, and in many respects, less complex than widefield, steady-state FRET microscopy. With steady-state FRET, the sensitized emission signal is contaminated by directly excited donor and acceptor fluorescence. Therefore, multiple control images must be collected, requiring acquisition times of at least approximately 3 seconds on optimized systems.(Vogel et al., Sci STKE 2006(331):re2 (2006); Berney et al., Biophys J 84(6):3992-4010 (2003); Gordon et al., Biophys J 74(5):2702-2713 (1998)). The controls are then mathematically processed to obtain a corrected FRET image that is then further normalized to account for the relative abundance of donors and acceptors present in the sample. Even with careful correction and normalization, signal changes of only approximately 10% are typically observed between FRET-positive samples and negative controls.(Kunkel et al., J Biol Chem 284(36):24653-24661 (2009); Llopis et al., Proc Natl Acad Sci USA 97(8):4363-4368 (2000)). In order to confidently assess whether small FRET signals reflect biologically relevant molecular interactions, the FRET efficiency must be determined by performing additional experiments such as acceptor photobleaching or measurement of FRET standards of known efficiency (Vogel et al., Sci STKE 2006(331):re2 (2006)). By contrast, TR-FRET imaging only detects signals emanating from interacting molecules. An approximate 4-fold change (300% increase) was seen in mean signal-to-noise ratio of terbium-sensitized GFP emission between cells expressing interacting and non-interacting ZO-1/PDZ1-eDHFR and GFP-cldn1/tail pairs. For signal-to-noise ratio calculations, images were used that were acquired at relatively long acquisition times (8 seconds, although intermolecular TR-FRET could be detected with acquisition times as short as 0.67 seconds). Thus, in addition to enhanced sensitivity and signal-to-noise ratio, TR-FRET displays increased time resolution that may allow analysis of interaction dynamics that cannot be resolved by traditional steady-state FRET or FRET-FLIM.

The results show that eDHFR fusion proteins can be specifically and stably labeled with a luminescent terbium complex, TMP-Lumi4, in living, wild-type mammalian cells. The ability to selectively impart terbium luminescence enables dynamic, non-destructive TR-FRET imaging of intracellular interactions between eDHFR and GFP fusion proteins without additional control measurements and mathematical processing. By detecting terbium-sensitized GFP emission at long lifetimes, detection of cellular autofluorescence, unbound terbium probe luminescence, and directly excited GFP fluorescence is effectively eliminated, thereby imaging TR-FRET in living cells at signal-to-noise ratios that exceed the observed FRET dynamic range of CFP and YFP fusion proteins in cell-free systems. Intermolecular TR-FRET image acquisition was substantially faster (<1 s) than steady-state FRET imaging (approximately 3 seconds) and orders of magnitude faster than FLIM imaging of FPs.

While the techniques described here should improve live-cell studies of protein function, there is broad scope for further enhancements and modifications. For instance, the large signal-to-noise ratio of intramolecular TR-FRET could be exploited to develop biosensors that incorporate eDHFR and GFP in a single fusion protein, analogous to CFP/YFP sensors designed to detect cellular analytes or enzyme activity.(Zhang et al., Nat Rev Mol Cell Biol 3(12):906-918 (2002)). Time-resolved microscopy can be easily adapted to measure donor or sensitized acceptor lifetimes by capturing and analyzing a series of images at different detector delay intervals.(Elangovan et al., J Microsc 205(Pt 1):3-14 (2002); Heyduk et al., Anal Biochem 289(1):60-67 (2001)). The multiple terbium emission peaks of TMP-Lumi4 can serve as a FRET donor to both GFP and red fluorescent proteins, potentially enabling the simultaneous detection of more than one molecular interaction in a single living cell. Finally, both SLO and pinocytosis probe delivery methods can be easily adapted for use in multiwell plate format, enabling high-throughput screening assays of intracellular protein-protein interactions using commercially available time-resolved fluorescence plate readers.

1. A composition comprising a lanthanide complex (LC) and a cell-penetrating peptide (CPP), the LC comprising a chelating moiety in association with a lanthanide and a ligand.

2. The composition of claim 1 wherein the ligand is trimethoprim (TMP).

3. The composition of claim 1 wherein the lanthanide is terbium.

4. The composition of claim 1 wherein the lanthanide is europium.

5. The composition of claim 1 wherein the cell-penetrating peptide is selected from the group consisting of oligo-arginine, a TAT-peptide, and rabies virus glycoprotein.

6. A method of labeling a first target molecule comprising:
contacting the target molecule with a non-toxic concentration of a lanthanide complex (LC) under conditions that allow association of the LC and the first target molecule, wherein the association labels the first target molecule, said LC comprising a ligand and a chelating moiety comprising a lanthanide.

7. The method of claim 6 wherein the LC associates with a first detector molecule fused to the target molecule.

8. The method of claim 7 wherein the first detector molecule associates with the LC through a ligand associated with the LC.

9. The method of claim 6 further comprising a second target molecule, the second target molecule fused to a second detector molecule, the method performed under conditions wherein the second target molecule interacts with the first target molecule.

10. The method of claim 9 wherein interaction of the first target molecule with the second target molecule brings the LC and the second detector molecule in to sufficient proximity to permit an energy exchange from the LC to the second detector molecule and wherein energy transfer to the second detector molecule permits detection of the first target molecule.

11. The method of claim 6 wherein labeling is in a host cell.

12. The method of claim 6 wherein labeling is on the surface of a host cell.

13. The method of claim 6 further comprising the step of culturing the host cell transformed or transfected with a first nucleic acid construct comprising a promoter element operatively-linked to a nucleic acid encoding a fusion protein, the fusion protein comprising the first target molecule and a first detector molecule under conditions such that a first target molecule/detector molecule fusion is expressed.

14. The method of claim 9 further comprising the step of culturing the host cell transformed or transfected with a second nucleic acid construct comprising a promoter element operatively-linked to a nucleic acid encoding a fusion protein, the fusion protein comprising the second target molecule and a second detector molecule under conditions such that a first target molecule/detector molecule fusion is expressed.

15. The method of claim 6 wherein the LC bound to the fusion protein is detected with a device.

16. The method of claim 6 wherein the lanthanide is terbium.

17. The method of claim 6 wherein the lanthanide is europium.

18. The method of claim 6 wherein the presence of the first target molecule is detected by fluorescence microscopy.

19. The method of claim 9 wherein the second detector molecule is a fluorophore.

20. The method of claim 19, wherein the fluorophore is a fluorescent protein.

21. The method of claim 6 wherein the first detector molecule is a dihydrofolate reductase and the ligand in the LC is trimethoprim (TMP).

22. The method of claim 6 wherein the first detector molecule is a dihydrofolate reductase and the ligand in the LC is a trimethoprim (TMP) analog.

23. The method of claim 6, wherein the first target molecule is selected from the group consisting of a protein, a protein domain, and a peptide.

24. The method of claim 9 wherein the second target molecule is selected from the group consisting of a protein, a protein domain, and a peptide.

25. The method of claim 6 wherein the LC further comprises a cell-penetrating peptide (CPP).

26. The method of claim 25 wherein the CPP is selected from the group consisting of oligo-arginine, a TAT-peptide, and rabies virus glycoprotein.
